(19) United States
(12) Patent Application Publication
(10) Pub. No.: US 2018/0194626 A1
(43) Pub. Date: Jul. 12, 2018

(54) Methods and Apparatus for Recovery of Volatile and Carbonaceous Components from Unconventional Feeds

(71) Applicant: Pioneer Astronautics, Lakewood, CO (US)

(72) Inventors: Mark Berggren, Golden, CO (US); Boris Nizamov, Highlands Ranch, CO (US); Robert Zubrin, Golden, CO (US); James Siebarth, Lakewood, CO (US)

(73) Assignee: Pioneer Astronautics, Lakewood, CO (US)

(21) Appl. No.: 15/861,557

(22) Filed: Jan. 3, 2018

Related U.S. Application Data
(60) Provisional application No. 62/442,410, filed on Jan. 4, 2017.

(51) Int. Cl. C01B 3/36; C10G 1/00; C07C 1/04; C07C 29/151; E21C 51/00; C25B 1/04

(52) U.S. Cl. CPC C01B 3/36; C10G 1/002; C07C 1/0485; C07C 29/1516; C01B 2203/1235; C25B 1/04; C01B 2203/0211; C01B 2203/062; C01B 2203/061; E21C 51/00

(57) ABSTRACT

A device to extract water and volatile organic compounds from asteroids, comets, and other space resources for propellant production, life support consumables, and manufacturing from in-situ resources in support of advanced space exploration is described. The device thermally extracts ice and water bound to clay minerals, which is then combined with small amounts of oxygen to gasify organic matter contained in carbonaceous chondrite asteroids. In addition to water, the device produces hydrogen, carbon monoxide, and carbon dioxide that comprise precursors to oxygen for propellant and breathing gas and organic compounds including fuels and plastics.

The device and methods are also applicable to the recovery of moisture, volatiles, and carbonaceous matter from low-grade terrestrial resources and waste materials. Application of the technology to terrestrial resources and wastes containing relatively low concentrations of carbonaceous matter is useful on Earth to obtain fuel components and water in an efficient manner. The technology enables the use of unconventional feed materials such as coal preparation waste, oil shale, contaminated soils, municipal wastes, and renewable resources and their byproducts produces valuable fuels and chemicals while mitigating detrimental environmental issues related to conventional storage or disposal of such materials.

### RELATED APPLICATIONS

This application claims the benefit of U.S. Provisional application No. 62442410 titled “Carbonaceous Asteroid Volatile Recovery System” filed Jan. 4, 2017 which is incorporated herein by reference.

### GOVERNMENT SUPPORT STATEMENT

This invention was made with Government support under National Aeronautics and Space Administration (NASA) JPL SBIR Contract No. NNX15CP30P (Phase I) and Contract No, NNX16CP21C (Phase II). The Government has certain rights in this invention.

### BACKGROUND OF THE INVENTION

It has been estimated that the mineral wealth resident in the belt of asteroids between the orbits of Mars and Jupiter would be equivalent to about 100 billion dollars for every person on Earth today. Many near-Earth asteroids with diameters up to 40 kilometers are present in orbits that approach or cross that of the Earth (Davis et al., 1993). Characteristics of these asteroids have been derived by inference from the composition of meteorites and from spectral analysis (Binzel et al., 1996). Chondrites represent more primitive forms of asteroids, and the subset of carbonaceous chondrites are highly oxidized and contain up to 20 percent bound water and up to 6 percent organic matter (Lewis and Hutson, 1993). This class of asteroid is thought to primarily contain water-bearing phyllosilicates, or clays (Nichols, 1993) with water present as bound water and hydroxyl groups. These asteroids also contain organic matter in the form of kerogen-like material (Kerridge, 1993), amino acids (Ehrenfreund et al., 2001), polycyclic aromatic hydrocarbons such as naphthalene (Plows et al., 2003), and other organic compounds. Carbon exists primarily in organic matter with only a small fraction as carbonates or more-refractory materials such as graphite and diamond (Sephton, 2002). The water and hydrocarbon contents of carbonaceous chondrite asteroids make them an attractive target for the recovery of resources valuable to human space exploration. Earth-based materials such as low-grade carbonaceous resources and organic-containing wastes exhibit similar properties and are suitable for processing in a manner similar to those discussed herein.

### SUMMARY OF THE INVENTION

A device to extract and recover water and organic matter to support propellant production, breathing gas, and life support, and manufacturing from in-situ resources in support of advanced space exploration is described. The device thermally extracts ice and water bound to clay minerals, which is then combined with small amounts of oxygen to gasify organic matter contained in carbonaceous chondrite asteroids. In addition to water, the device produces hydrogen, carbon monoxide, and carbon dioxide that comprise precursors to oxygen for propellant and breathing gas and organic compounds including fuels and plastics. Additional device byproducts include nitrogen, sulfur, and phosphorus compounds extracted from asteroid material that have potential uses as buffer gas for life support and reagents for more-advanced asteroid materials processing. The device and methods are also applicable to the recovery of moisture, volatiles, and carbonaceous matter from low-grade terrestrial resources and waste materials. Application of the device to terrestrial resources and wastes containing relatively low concentrations of carbonaceous matter is useful on Earth to obtain fuel components and water in an efficient manner. The technology enables the use of unconventional feed materials such as coal preparation waste, oil shale, contaminated soils, municipal wastes, and renewable resources and their byproducts produces valuable fuels and chemicals while mitigating detrimental environmental issues related to conventional storage or disposal of such materials.

### BRIEF DESCRIPTION OF DRAWINGS

FIG. 1. Carbonaceous Asteroid Volatile Recovery System flow sheet.

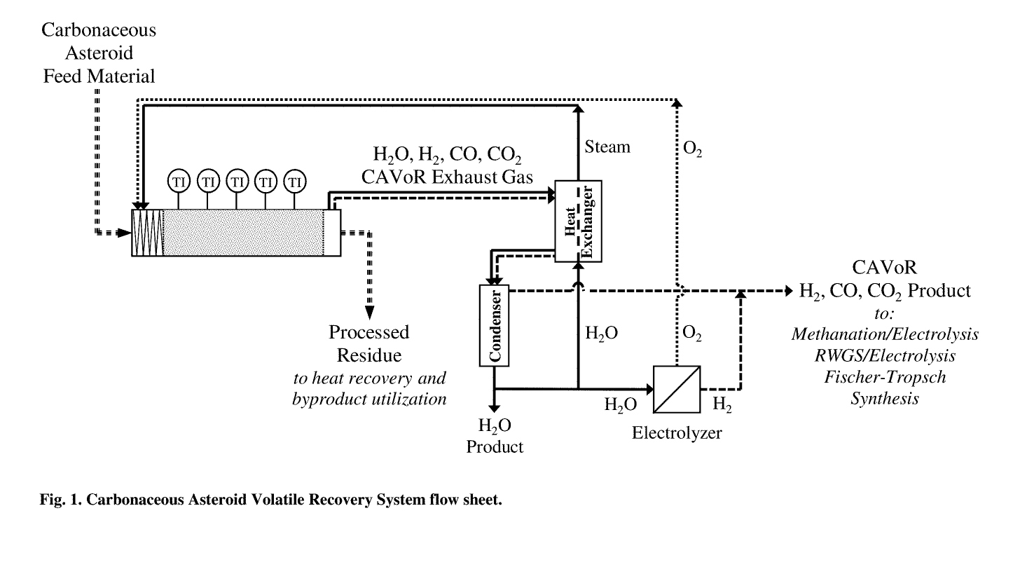

FIG. 2. Thermogravimetric analysis of as-received organic simulant constituents.

FIG. 3. Thermogravimetric analysis of organic simulant constituents; ash-free basis.

FIG. 4. CAVoR R3 reforming summary—Gilsonite simulant.

FIG. 5. CAVoR R6 reforming summary—oil sands simulant.

FIG. 6. CAVoR R7 reforming summary—petroleum coke simulant.

FIG. 7. CAVoR R11 reforming summary—oil shale simulant.

FIG. 8. CAVoR reforming at reduced oxygen ratio (Gilsonite simulant).

FIG. 9. CAVoR-methanation-electrolysis summary material balance.

FIG. 10. CAVoR-methanation-electrolysis model block flow diagram.

FIG. 11. Pioneer conceptual continuous-flow CAVoR reforming reactor.

FIG. 12. CAVoR reforming reactor illustrations.

### DETAILED DESCRIPTION OF THE INVENTION

Comets and asteroids are objects that can be exploited for their valuable raw materials. In one embodiment, a device and processes to mine these minerals and then bring them back to Earth is described. In one embodiment, the claims describe an efficient system to mine materials for use as propellants in space vehicles. In one embodiment, the claims describe an efficient system to mine materials for use in fabricating equipment for space vehicles. In one embodiment, a device for recovery of carbonaceous and volatile matter from low-grade Earth resources such as high-ash coal and oil shale for the purposes of fuel production is described. In one embodiment, a device and processes for recovery of resources from wastes while rendering solid residues sterile and safe for disposal and use as filler in concrete and other materials is described.

In one embodiment, a system for recovering materials used in developing the space structures and in generating the rocket fuel that will be required to explore and colonize our solar system is described. Asteroids are rich in the mineral raw materials required to build structures in space and in many cases contain significant concentrations of moisture and organic matter. The moisture and organic matter is contained in regolith near the surface or at modest depths. Regolith consists of loose particles or deposits generally covering underlying rocky material. It includes dust, soil, broken rock, and other related material Comets are also rich resources for the water and carbon-based molecules necessary to sustain life. In addition, an abundant supply of cometary water ice could provide copious quantities of liquid hydrogen and oxygen, two primary ingredients in rocket fuel. In one embodiment there is provided a system for materials recovery from asteroids to provide the raw materials for space structures.

In one embodiment of the invention, an auger reactor for continuous processing of regolith or terrestrial feeds is described. In one embodiment, heat is recovered from hot gases and solids and is transferred to the feed gases and solids. In one embodiment of the invention, a dual fixed-bed reactor system in which batch processes are carried out in a sequence in which heat from processed residue in one reactor is directly or indirectly transferred to fresh solid and gas feeds for the second reactor is described.

Water can be recovered from hydrated clays upon heating. The required temperature and rate of moisture recovery greatly depends on the mineral structure of clay. However, initial indications of water removal from terrestrial clays are observed at temperatures well below 200° C. while the majority of water from many types of clay is removed at temperatures below 500° C. Efforts have been directed toward the dehydration and decomposition characteristics of phyllosilicates relevant to exploitation of asteroidal materials. Much of the organic matter in terrestrial oil shale, which contains a significant portion of kerogen-like material, can be extracted via pyrolysis at temperatures in the 400 to 500° C. range. Because the process is typically carried out at temperatures in the 700-800° C. range, the great majority of water can be recovered from the target resource.

A preferred process approach for asteroid materials containing analogous organic matter is the use of non-catalytic steam reformation and partial oxidation to maximize hydrocarbon recovery while producing syngas that can be further processed (via catalytic or non-catalytic thermochemical reactions and electrolysis) to generate oxygen, chemicals, and fuels useful for propulsion and manufacturing. Such an approach allows for the effective removal and recovery of byproducts such as nitrogen (via dissociation of ammonia, cyanide compounds, and other gaseous contaminants) and sulfur (via sorption of compounds from the gas phase followed by concentration and recovery during sorbent regeneration).

Steam reformation was used historically to produce synthesis gas (called “town gas” at the time) from coal via reaction (1).

C+H₂O(g)=>CO+H₂ΔH=+136kJ@750° C. (1)

If more steam is available, the CO will be converted to CO₂ via the water gas shift reaction (2).

CO+H₂O=>CO₂+H₂ΔH=−35kJ@750° C. (2)

Taken together, the net result of reactions (1) and (2) is reaction (3).

C+2H₂O=>CO₂+2H₂ΔH=+101kJ@750° C. (3)

Similarly, steam reformation can be used to produce mixtures of CO₂, H₂, and CO from organic material. For example, we can steam reform methane via reaction (4).

CH₄+2H₂O=>CO₂+4H₂ΔH=+190kJ@750° C. (4)

Steam reformation of all kinds of organic matter can be readily performed. For example, naphthalene (representing a segment of kerogen-like substance) is steam reformed via reaction (5).

C₁₀H₈(g)+20H₂O=>10CO₂+24H₂ΔH=+884kJ@750° C. (5)

In one embodiment, the overall endothermic steam reformation reactions shown above require significant energy input, which can be supplied via external sources such as resistive heaters but can also be supplied at least in part by small additions of oxygen. In this application, oxygen also aids the decomposition of more-complex organic molecules, which leads to greater recovery of carbon at faster rates with lower risk of tar formation. The partial oxidation of carbon to carbon monoxide is shown in the following equation.

C+½O₂=COΔH=−112kJ@750° C. (6)

More complex compounds can also be subjected to partial oxidation, as shown by the example for naphthalene in equation (7).

C₁₀H₈(g)+5O₂=10CO+4H₂ΔH=−1249kJ@750° C. (7)

Carbon monoxide formed as shown in the partial oxidation equations above can be further oxidized to carbon dioxide with significant heat release as shown below.

CO+½O₂=CO₂ΔH=−283kJ@750° C. (8)

In one embodiment, autothermal steam reforming (which utilizes a mixture of oxygen and steam) results in gas mixtures containing hydrogen, carbon monoxide, and carbon dioxide (along with excess water) depending on the steam and oxygen ratios and operating temperatures, which are important factors in establishing the thermodynamic equilibrium. In typical applications of autothermal steam reforming, a 2:1 steam:carbon (molar) ratio is used along with a 0.5:1 oxygen:carbon (molar) ratio. In other embodiments, the amount of water present in carbonaceous asteroids of the composition shown above is sufficient to supply the needs for reforming as well as electrolysis to produce oxygen for partial oxidation.

In some embodiments of the claims, the process employs in-situ resource utilization leveraging resources found or manufactured on other astronomical objects (the Moon, Mars, asteroids, etc.) to fulfill or enhance the requirements and capabilities of the system. In some embodiments, the process employs terrestrial resources to recover volatile and carbonaceous matter useful for production of fuels and chemicals.

### Experimental

Although the following experimental procedures are described in detail, they are illustrative and not limitative of the reminder of the description. The reagents used in preparing these compounds are either available from commercial vendors and chemical suppliers such as SigmaAldrich, Alfa Aesar, TCI, etc. or are prepared by methods well known to a person of ordinary skill in the art, following procedures described in literature. Samples were analyzed using GC, EDS Analysis on SEM and x-ray fluorescence (XRF).

A preliminary rough projection of the results of autothermal reforming of organic matter in asteroids was prepared using naphthalene as an analog chemical. HSC Chemistry® software was used to perform the thermodynamic calculations resulting in the projected results. The organic matter in asteroids likely consists of longer-chain hydrocarbons containing small amounts of oxygen, but the present example serves to illustrate the overall thermodynamics of autothermal steam reforming under relevant conditions. Experiments were carried out with similar reactions on plastic polymers and other complex organic compounds with virtually complete conversion. One mole of naphthalene (C₁₀H₈) reacted with 20 moles of water and 5 moles of oxygen results in the following equilibrium gas mixtures at temperatures of 500 and 750° C. at one bar pressure as shown below. (Naphthalene is entirely decomposed at temperatures above about 300° C.).

TABLE 1
Thermodynamic Equilibrium of
Autothermal Steam Reforming of Naphthalene.

| | Reaction Temperature, ° C. | | | |
|---|---|---|---|---|
| Equilibrium | 500° C. | | 750° C. | |
| Composition | Wet | Dry | Wet | Dry |
| Vol % H₂O | 34.4 | — | 40.3 | — |
| Vol % CO | 5.0 | 7.6 | 10.9 | 18.3 |
| Vol % CO₂ | 24.4 | 37.2 | 18.5 | 31.0 |
| Vol % H₂ | 36.2 | 55.2 | 30.3 | 50.8 |

| | | |
|---|---|---|
| H₂:CO | 7.2:1 | 2.8:1 |
| CO₂:CO | 4.9:1 | 1.7:1 |

The equilibrium results in the table above incorporate the individual partial oxidation, steam reforming, and water gas shift reactions discussed earlier. Further calculations showed that significant variations of the key input parameters (oxygen ratio, steam ratio, temperature, and pressure) are possible while still achieving virtually complete conversion of organic matter to syngas. Additional water gas shift or reverse water gas shift reactions can be performed as the product gas cools or after condensing and removing excess water from the product gas, resulting in the ability to target specific H_{2:}CO ratios for downstream fuels or chemicals synthesis.

Autothermal reforming carried out with hydrocarbon feeds that contain only small amounts of inorganic or inert matter (where the reaction temperature is controlled by the steam and oxygen feed ratios to balance partial oxidation with reforming at the target operating temperature) require no additional heat input after preheating. In addition preheating requirements from normal terrestrial ambient temperatures are lower than those for asteroid materials that may be at significantly lower initial temperatures. In the case of asteroid materials that contain majority inorganic matter along with bound water or hydroxyl groups and much smaller proportions of hydrocarbons, a somewhat different approach is required. This is largely due to the significant amount of heat retained in the processed residue, which is not carried with the exhaust gas and is not as readily available to exchange against steam and oxygen inputs. However, as discussed later, several techniques are available to recover heat from the processed residue and to use heat from downstream exothermic reactions such as methanation and fuels synthesis to preheat the asteroid materials prior to autothermal steam reforming. Efficient process heat exchange is further enabled by the large delta T applied to cold asteroid materials.

A preliminary energy balance was projected for simulated asteroid materials containing 15 percent (by weight) water, 5 percent organic matter, and balance clay to establish rough estimates and distribution of energy inputs to raise the temperature of the asteroid material from −73° C. (at the lower range of asteroid surface temperatures) to 750° C. (typical reforming inlet temperature). Included are melting and vaporization of water and hydrocarbon components. Water in the form of ice was assumed for these simplified calculations, and naphthalene was used to represent the organic matter. These heat calculations do not include partial oxidation or reforming reactions, which are discussed later. The following table summarizes the thermal heating, melting, and vaporization requirements for the example case.

TABLE 1
Thermal Energy Requirements for Heating Asteroid Feed Materials.
(1 kg Asteroid Material containing 15 wt % H₂O, 5% Organic Matter,
and Balance Inorganics; Temperature Raised from −73° C. to 750° C.)

| | Thermal Energy Input, kJ | | | |
|---|---|---|---|---|
| | per 1 kg Asteroid Material | | | |
| Thermal Energy Parameter | Water | Organics | Inorganics | Total |
| Raise Temperature of | 281 | 72 | 909 | 1,262 |
| Solid, Liquid, and/or Gas | | | | |
| Heat of Fusion | 50 | 7 | — | 57 |
| Heat of Vaporization | 341 | 28 | — | 369 |
| Total | 672 | 107 | 909 | 1,688 |

The net heat release from autothermal reforming of 50 grams of organic matter shown in Table 2 above (contained in one kilogram of asteroid material) to the indicated equilibrium composition at 750° C. using a 2:1 steam:carbon ratio and a 0.5:1 oxygen:carbon ratio is about 570 kJ. This is equivalent to roughly one-third of the required thermal heat input shown in Table 2. Much of the required heat input can be recovered from reaction products. For example, one-half of the heat contained in the inorganic residue (about 450 kJ) can be captured by indirect heat exchange of the residue against fresh feed. A more-sophisticated system with a multi-cell thermal battery and a counter-flow regolith heat exchange system can achieve up to 80 percent heat recovery. Further, the system can be designed for two reactors, allowing residual heat from one reactor to be exchanged to steam fed to the second reactor. Additional useful heat recovery can be obtained by indirect exchange of the hot exhaust gases to the asteroid feed material or to other feeds such as oxygen and water. Therefore, the vast majority of the required thermal energy input for pre-heating can be provided by heat recovery and partial oxidation reactions.

Oxygen was added to aid the breakdown of more-complex organic molecules during autothermal reforming. The 0.5:1 oxygen:carbon ratio stated above is probably near the upper limit needed to ensure this requirement (and as shown, would generate thermal energy useful to sustain reforming in the presence of inorganic matter). If alternate non-chemical, non-electric heat sources were used, the oxygen addition could likely be reduced significantly. However, for a conservative case, a 0.5:1 O₂:carbon molar ratio was used to determine electrolysis requirements for the same one kilogram asteroid material case shown above.

At the specified 50 grams of organic matter contained in one kilogram of asteroid material, 62.4 grams of oxygen would be required to satisfy the 0.5:1 oxygen:carbon molar ratio. Electrolysis of liquid water to produce the required amount of oxygen would require 1,115 kJ of electrical energy (at 100% efficiency) or about 1,400 kJ at 80% efficiency. Additional hydrogen would be co-produced with oxygen, which enhances the ability to perform downstream fuels and chemicals synthesis.

The integration of autothermal reforming with oxygen production via electrolysis is shown from the two equations below (using naphthalene as the model organic compound and the thermodynamic equilibrium conversion results shown above in Table 1.

Autothermal reforming equilibrium result:

C₁₀H₈(g)+5O₂+20H₂O(g)=>3.7CO+13.7H₂O(g)+6.3CO₂+10.3H₂ΔH=−1467kJ(thermal)@750° C. (9)

### Electrolysis:

10H₂O=>10H₂+5O₂ΔH=+2858kJ(electric)@STP (10)

The overall system result is Equations (9) plus (10):

C₁₀H₈(g)+16.3H₂O=>3.7CO+6.3CO₂+20.3H₂ (11)

It is important to note that the amount of water present in carbonaceous asteroids of the composition shown above is sufficient to supply the needs for reforming as well as needs for electrolysis to produce oxygen for partial oxidation.

Assuming an overall average processing rate of one kilogram per hour of carbonaceous chondrite material of the composition stated above, the electrolysis power requirement for the system, herein described as the Carbonaceous Asteroid Volatile Recovery System (CAVoR) is about 310 watts (at 100 percent efficiency) or about 390 watts at 80 percent efficiency to supply the required oxygen for autothermal reforming. The thermal input is largely provided by partial oxidation, downstream fuels or chemical synthesis waste heat, and heat recovery from process residues.

This example shows the system up to the point where further processing would be carried out to manufacture organic compounds and oxygen from the reaction products. Further processing could include reverse water gas shift (RWGS) to convert CO₂ to CO and to adjust the H_{2:}CO ratio, Sabatier/electrolysis to produce methane and oxygen, methanol synthesis (for fuel or subsequent olefin and plastic synthesis, Fischer-Tropsch reactions to produce hydrocarbon fuels, or direct olefins synthesis from syngas. The exact outcome for each process route depends on the carbon, hydrogen, and oxygen proportions derived from water and organic compounds present in asteroid feed materials. The following example describes conversion of the system output to methane and oxygen.

### Methanation/Electrolysis of CAVoR Product

In this example, the CAVoR product as described above is subjected to methanation to convert CO and CO₂ to methane and water according to the following reactions.

CO₂+4H₂=>CH₄+2H₂O(g)ΔH=−181kJ@400° C. (12)

CO+3H₂=>CH₄+H₂O(g)ΔH=−219kJ@400° C. (13)

Hydrogen produced by the system is used in the above methanation reactions. Additional required hydrogen is obtained by electrolysis of the water produced by methanation, along with some of the water produced directly by the system. Electrolysis proceeds according to the following reaction.

H₂O(l)=>H₂+½O₂ΔH=286kJ@STP (14)

After all of the CO and CO₂ in the system exhaust gas is converted to methane, and electrolysis is performed to supplement the hydrogen produced in the system, the following composition is obtained.

TABLE 2
Methanation/Electrolysis of CAVoR Product
(based on 1 kg carbonaceous chondrite containing
15% (wt) H₂O and 5% (wt) organic matter.

| Methanation/ | | | |
|---|---|---|---|
| Electrolysis | Methanation/Electrolysis Product Composition | | |
| Constituent | Mol % | Wt % | Mass, g |
| H₂O | 22.8 | 18.6 | 37.3 |
| CH₄ | 42.8 | 31.2 | 62.4 |
| O₂ | 34.4 | 50.2 | 100.3 |
| Total | 100.0 | 100.0 | 200.0 |

The electrolysis requirement for methanation/electrolysis of the CAVoR product is about 500 watts (at 100 percent efficiency) or about 620 watts at 80% efficiency assuming an operating rate of 1 kg carbonaceous chondrite material per hour. Therefore, the total electrolysis power for a CAVoR/methanation/electrolysis system is about 810 watts (at 100 percent efficiency) to produce about 1.5 kg/day methane, 2.4 kg/day oxygen, and 0.9 kg/day water. Additional electrical requirements for pumps, instruments, and other ancillary hardware are expected to be low relative to electrolysis power.

It must be noted that one significant benefit of the CAVoR combined steam-oxygen system to gasify hydrocarbons from asteroid materials is that much of the hydrogen required in syngas is formed thermo-chemically without the use of electricity-intensive electrolysis of water. For an alternative system in which the same organic matter in the asteroid is simply oxidized to produce CO plus H₂O followed by methanation/electrolysis, the total electrolysis power would be 1.36 kilowatts (assuming 100 percent efficiency) versus only 0.81 kilowatts for the CAVoR/methanation/electrolysis scenario, which represents a reduction of 40 percent in electrolysis power.

The modeling of mass and energy balances for CAVoR coupled with downstream methanation-electrolysis defines the thermal and electrical energy loads and power inputs resulting from target production rates. Thermal heat inputs and efficient heat recovery methods can be identified and evaluated for each of the integrated CAVoR scenarios.

### Conceptual CAVoR System

Because no standardized carbonaceous chondrite simulant exists, suitable terrestrial analogs including matrices consisting of phyllosilicate clays exhibiting lightly-bound water, strongly-bound water, and water recoverable from hydroxyl groups were incorporated to produce representative feeds. In some cases, water was infused into the matrices at specified concentrations. Organic matter such as that present in terrestrial oil shale appears to be a reasonable initial starting material. Such organic matter may be incorporated in the form of crushed or ground oil shale and as extracted kerogen-like material infused into clays. Smaller known amounts of specific compounds such as magnetite were used to establish effects of iron compounds known to exist on carbonaceous asteroids on the CAVoR process results. Systematic preparation and testing of these terrestrial analogs was used to establish important chemical and materials handling factors. The asteroidal simulants are also representative of some Earth-based resources such as coal processing wastes.

Typical steam reformer feeds contain only small amount of inorganic matter. Therefore, nearly all of the feed material is converted to gas. In Pioneer's Lunar Organic Waste Reformer, which processes organic material containing only small amount of inorganic matter, the organic feed is pushed through a static reaction zone where steam and oxygen are injected and rapidly consumed. In a batch operation, the system is stopped when only a small amount of organic matter remains just upstream of the reaction zone. In Pioneer's initial batch CAVoR reactor, much of the feed volume (consisting of inorganic matter) remains in the reactor after removing water as steam and converting organic matter to gas. In one potential implementation, a fixed-bed of relatively high length:diameter ratio is subjected to autothermal steam reforming via injection of steam and oxygen at the feed-end of the reactor, resulting in a reaction front that moves along the length of the reactor creating a hot zone at the location of the steam-oxygen reactions. A spring or plunger system is keeps the material compacted to the desired degree (and accommodates microgravity conditions) while the volume associated with water and the organic fraction are removed as gases. A series of thermocouples along the length of the reactor (along with gas sensors on the exhaust gas stream) signal when the reaction is complete. The initial CAVoR system was configured to run in batch operation mode. Batch operations simplify feeding and residue transfers while allowing for durable stationary seals.

### Contaminant Removal and Byproduct Recovery

During CAVoR processing, sulfur would likely be released from both organic and inorganic sources mostly in the form of H₂S and COS. Sulfur needs to be removed prior to any downstream synthesis steps to prevent catalyst deactivation or poisoning. The simplest approach (used in the initial reactor system) is to install a zinc oxide or other suitable sulfur trap in partially-cooled CAVoR system exhaust gas piping. Zinc oxide is not typically regenerated for reuse or recovery of byproduct sulfur, but it is capable of reducing sulfur concentrations to sub ppm levels and can also be used as a backup and polishing sorbent downstream of a regenerable sorbent. A non-regenerable alkali aluminate sorbent can be used to trap halide gases as needed. Separately, Pioneer developed a cobalt-based regenerable sulfur sorbent system for lunar or Mars ISRU applications that incorporates an efficient reductive-regeneration system to recover concentrated sulfur and to prepare sorbent for re-use. A regenerable manganese-based sorbent can be used to remove halides. Pioneer has also developed other methods using in situ resources such as the iron and base compounds in lunar regolith to trap sulfur and halides—these concepts may also be applicable to more-advanced CAVoR implementation.

Tars may be formed or released during preheating. Pioneer's previous experience indicates that tars can be destroyed using olivine, metallic catalysts, or high temperatures in the presence of steam. The maintenance of a small hot zone just downstream of the CAVoR reactor is expected to convert tars to syngas during heating or other transient conditions. The hot zone can be created from thermal or electrical input or by slight addition of oxygen or steam.

Nitrogen is present in primarily in the organic matter of carbonaceous chondrites and is released primarily as N₂ gas (Nichols, 1993). Opportunities exist to recover nitrogen (useful as buffer gas or pressurant) at various stages of the CAVoR process, particularly at locations with minimal CO concentration or at locations after CO is reacted to a liquid or solid product. Small amounts of nitrogen may be released during CAVoR as ammonia, which can be decomposed in the CAVoR exhaust stream over a tungsten, rhodium, or other suitable catalyst, releasing nitrogen and hydrogen and preventing water contamination issues.

Phosphorus is present in small amounts in both inorganic and organic constituents of carbonaceous chondrites (Pasek et al, 2004). Some phosphorus may be released during the CAVoR thermochemical process as reduced gaseous compounds.

Carbonaceous chondrite asteroids contain iron in many forms, including magnetite and other oxidized or partially oxidized forms within silicates and other minerals. A preliminary thermodynamic analysis of magnetite in the presence of the equilibrium CAVoR product gas shows some potential for partial reduction of magnetite to FeO and Fe, primarily via conversion of hydrogen to steam. The opportunity for recovery of iron-enriched minerals from the CAVoR residue is enhanced by the removal of organic matter and water. Previous lunar ISRU work at Pioneer has shown the potential to concentrate liberated iron-bearing minerals via centrifugal, magnetic, and electrostatic methods in vacuum.

Carbonaceous chondrites consist of weak, friable particles in a clay mineral matrix (O'Neill, 1979). These characteristics imply that minimal crushing and grinding will be required prior to processing. The CAVoR residue is expected to be more free-flowing than the feed following removal of moisture and organic matter. The relatively low CAVoR operating temperature should eliminate the risk of mineral fusion. The processed residue should have potential for use as feed to mineral separations, bulk shielding, and feed to higher-temperature fusion processes to fabricate formed shapes.

### Autothermal Steam Reforming Reactor

A reactor was designed to accommodate batches of about 200 grams of asteroid simulant. This scale was determined to be suitable for screening a range of asteroid compositions while producing sufficient amounts of products to allow for the determination of material balances and system performance. A nominal 200-gram feed batch containing 15 percent total moisture and 5 percent organic matter would yield a theoretical 30 grams of water and 10 grams of hydrocarbons, not including weight changes due to oxygen and steam additions. For organic matter composed of pure carbon, an oxygen flow of 156 standard cubic centimeters per minute (sccm) is needed over a period of one hour when fed at a 0.5:1 oxygen:carbon molar ratio assuming a product gas containing a 2:1 molar ratio of CO_{2:}CO.

The initial reforming reaction conditions were based on conversion of hydrocarbons in a one-hour period. Based on previous work on a Lunar Organic Waste Reformer (Carrera et al., 2013), nominal molar ratios of 0.5:1 O₂:C and 2:1 H₂O:C were chosen using the assumption that much of the water present in the asteroid sample would be removed during pre-heating before reforming reactions start (i.e. water fed to the reactor would be independent of any water present in the sample). Therefore, water would be injected through a vaporizer at a rate of about 0.5 grams per minute over a one-hour reaction period (assuming a pure carbon feed) to accommodate a 2:1 H₂O:C ratio. These initial nominal target design flow rates were revised as experimentation proceeded.

The oxygen rate can strongly influence temperatures and reaction rates in the reforming zone and can determine the product CO₂:CO ratio. A higher H₂O:C ratio provides a driving force for the reforming reaction but can be reduced to increase temperature in the reaction zone. The temperature at which bound water in the asteroid feed material is released impacts the set point for the injected water rate. Because the asteroid feed compositions contain a minority of reactive material (and a significant mass of inert material that can absorb heat), the minimum amount of water necessary to provide the maximum reaction rate with minimal oxygen consumption was used. Oxygen rates were set to facilitate tracking of the reaction front through the reactor (by noting the position of the partial oxidation exothermic temperature rise as experiments proceeded). In general, oxygen flow was set with the goal of maximizing the steam reforming reactions with carbon and to maximize hydrogen yield useful for downstream methanation reactions.

A sample support consisting of a ceramic foam disc with an alumina felt filter was used to retain the asteroid simulant. A guard bed of granular carbon was placed in the bottom (exhaust zone) of the reactor to ensure that any oxygen that may bypass reactive hydrocarbons is consumed prior to exhausting the reactor system. The reactor was filled and discharged through a single, stationary seal at the upper reactor port. A pneumatic sample transfer system was employed to removed simulant residue following each experiment.

The reactor was constructed from a 12.5-inch (31.8-cm) length of 1.5-inch (3.81-cm) outside diameter polished 316 stainless steel tubing of 0.065-inch (0.165-cm) wall thickness. The inlet and outlet were fabricated from 1-inch (2.54-cm) Swagelok® fittings to allow for repeated filling, sealing, and discharge of reactor contents. A steam/oxygen injection port was installed near the top of the reactor to allow for close-coupling of the vaporizer subsystem. Eight ⅛-inch (0.318-cm) diameter thermocouples fittings were installed along the length of the reactor to facilitate characterization of the reaction front during experiments. Thermocouples were inserted to about one-fourth of the reactor diameter. The design projected that nearly complete oxygen consumption would be achieved with associated exotherms in a plug flow manner as the reaction proceeds from top to bottom. Therefore, a moving zone of higher temperatures was expected from the inlet to the outlet of the reactor as carbonaceous matter is reacted.

Provisions for an inert gas purge were installed above the reactor along with a pressure relief valve, inlet pressure measurement, and reactor inlet temperature measurement. A supplemental oxygen injection port was installed in the lower section of the reactor for possible use in converting organic matter that may pyrolyze and vaporize during the batch preheating portion of experiments.

The reactor was designed to allow its placement in a single, full-length radiant pre-heating furnace. However, upon installation two separate external heating zones were employed (via high temperature heat tapes and controllers with external reactor thermocouples) to permit different temperature zones as desired.

### Reactor Inlet Subsystem

A reactor inlet subsystem was designed and fabricated for placement above the reactor solids feed port. The inlet subsystem consists of a relief valve (Swagelok® SS-RL4S8) with a 0.5-inch (1.27-cm) diameter fitting for placement in a clear path above the reactor. A 1/16-inch (0.158-cm) diameter thermocouple was installed on a side port of a ½-inch diameter cross. A pressure gauge (0-30 psig) and a transducer (Omegadyne® PX209-030G5V) were installed on a ¼-inch cross attached to the other side port of the ½-inch diameter cross. The ¼-inch cross also contains a port for introduction of inert or process sweep gas, which also served as a tracer for flow rates and material balance purposes. The inert gas was manually fed through a metering valve and monitored using a mass flow meter (Aalborg® GFM17; 0-100 sccm). The inlet subsystem attaches to the reactor feed/discharge port and was removed between each test.

### Steam and Oxygen Injection Subsystem

Water was pre-heated and vaporized prior to injection into the down-flow fixed-bed reactor. Oxygen was used to sweep steam into a side port near the top of the reactor. The vaporizer consists of a ¾-inch (1.91-cm) Swagelok® tee into which a cartridge heater was installed (Watlow Firerod® ¼-inch outside diameter by 4-inches long; 300 watts at 120 volts; internal K thermocouple). Water was injected at a controlled, steady rate via a syringe pump (New Era® Model NE-300). Water was delivered through a 1/16-inch (0.159-cm) diameter tube inserted through a fitting above the vaporizer cartridge heater. Oxygen was metered through a 48-inch (122-cm) length of 1/16-inch outside diameter, small-bore stainless steel tube of 0.010-inch (0.025-cm) inside diameter. The flow rate was controlled by adjusting the upstream pressure. This system allowed for precise control of oxygen flow while preventing surges or excess flows that could result in runaway reaction or excessive exotherms. The oxygen was delivered from a commercial compressed gas cylinder through a pressure regulator and filter upstream of the flow-control tubing. A pressure transducer (Measurement Specialties® M5131-000005-100PG) was installed in this section of tubing for determination of flow via the data acquisition system and internal calculations.

Oxygen flow was calibrated through the flow control tubing based on transducer pressure readings (0.5 to 4.5 volts) and the pressure drop through the tubing (up to about 100 psi) to provide up to about 330 standard cubic centimeters per minute of oxygen.

The oxygen flow calibration shows that a precise flow is obtained over the range of interest for completion of batch reactions in time periods between 20 and 60 minutes (104 to 312 sccm oxygen for the nominal design condition) using regulated feed pressures between about 35 and 85 psig. The oxygen flow control tubing can be readily replaced to allow for flows greater or smaller than those used for initial testing. However, experiments run during initial work showed the as-installed tubing to be suitable for flows as low as 20 sccm oxygen and up to the maximum total flow of about 330 sccm.

### Condenser and Exhaust Gas Subsystem

A reactor exhaust cooling and condensing system was installed just downstream of the reactor to remove water prior to dry gas flow rate and composition measurements. The condenser system was submerged in an ice-water bath during tests. The condensate resulting from autothermal reforming was periodically withdrawn by applying slight system backpressure to determine its incremental rate during operations.

Periodic gas chromatograph analyses for hydrogen, carbon monoxide, carbon dioxide, methane, nitrogen, and other gas constituents were carried out during startup and operation. A separate gas chromatograph was used to monitor sulfur compounds released during processing. In addition, sensors for continuous measurement of carbon dioxide and methane were made using an e2V® IR-EK2 infrared sensor for measurements of concentrations ranging from 0 to 100 percent by volume. For safety purposes, a continuous oxygen sensor (Advanced Micro Systems Model P-3) was installed to detect the presence of unreacted oxygen in the exhaust. The oxygen sensor can read up to 25 percent oxygen to facilitate calibration using ambient air, but it can detect concentrations as low as about 0.1 percent. This sensor can help to determine whether oxygen has bypassed organic matter or whether temperature profile sensing failed to detect the end of the reaction. A sulfur sorbent column (48 grams of zinc oxide; Johnson Matthey HiFUEL A310® pellets) was installed following initial experiments to trap sulfur upstream of the oxygen sensor. Sampling ports were installed just upstream and downstream of the zinc oxide column to allow the sulfur trap performance to be evaluated.

A mass flow meter (Omega® FMA 1714; 0-1000 sccm) was installed to continuously measure the dry exhaust gas flow. This was used to indicate the onset of non-condensable gas release during pre-heating. Additionally, the flow measurement was used to track overall reaction rates. In combination with gas analyses, the continuous flow measurement was used to establish mass balances for each experiment. Additional periodic exhaust gas flow measurements were made manually using a bubble meter and were calculated from the known flow of helium sweep gas and its concentration in the dry exhaust gas.

### Integrated CAVoR System

The subsystems described above were integrated in preparation for installation of remaining interconnecting tubing as well as controls and instrumentation

### Data Acquisition System

A LabJack® U6 data acquisition module with a CB37 terminal board was used to read and log temperature, pressure, and flow data during experiments. DAQFactory® software was configured to manage the hardware-computer interface. Experimental data was monitored in real time and logged in intervals suitable for later analysis of test results. The data acquisition channel assignments and calibrations are shown in the following table.

TABLE 3
Data Acquisition System Channel Assignments.

| Channel | Channel | Channel | Calibration | Instrument |
|---|---|---|---|---|
| # | Tag | Description | Info | Info |
| 0 | O2 Flow | O2 Inlet Flow Meter | sccm O2 = −1.5535 v\hat{ }3 + | MSI M5131-000005-100 PG Transducer |
| | | | 20.8151 v\hat{ }2 + 21.4196 v − | (0.5-4.5 v out @ 5 vdc input) |
| | | | 16.4200 (r\hat{ }2 = 1.0000; | Flow calc based on ΔP through 48″ length |
| | | | nominal 0-360 sccm O2 span) | of 0.010″ ID Tube |
| 1 | He Flow | He Inlet Flow Meter | sccm He = 27.3389 v + | Aalborg GFM17; SN 15225 with |
| | | | 0.6110 (r\hat{ }2 = 0.9993; nominal | Display #1 (0-5 v out @ 12 vdc input) |
| | | | 0-137 sccm He span) | (factory delivered as 0-100 sccm H2) |
| 2 | Ex Flow | Exhaust Flow Meter | sccm (as He) = 298.7811 v − | Omega FMA 1714-60 psi; SN 76729-1 H2 |
| | | | 5.6569 (r\hat{ }2 = 0.9999; | (0-5 v out @ 12 vdc input) |
| | | | nominal 0-1488 sccm | (factory delivered as 0-1000 sccm H2) |
| | | | He span) | |
| 3 | Ex % O2 | Exhaust O2 Sensor | % O2 = 11.442623 v | AMI Model 60 with P3 Electrochemical Sensor |
| | | | (linear calibration using | (0-2.5 v out @ 12 vdc input) |
| | | | He and air) | factory delivered as 0-25% O2) |
| 4 | VT1 | Internal Vaporizor Temp | K-Thermocouple | Ungrounded 1/16″ TC |
| 5 | RTIN | Reactor Inlet Temp | K-Thermocouple | Ungrounded 1/16″ TC |
| 6 | RT1 | Reactor Temp 1 | K-Thermocouple | Ungrounded ⅛″ TC |
| 7 | RT2 | Reactor Temp 2 | K-Thermocouple | Ungrounded ⅛″ TC |
| 8 | RT3 | Reactor Temp 3 | K-Thermocouple | Ungrounded ⅛″ TC |
| 9 | RT4 | Reactor Temp 4 | K-Thermocouple | Ungrounded ⅛″ TC |
| 10 | RT5 | Reactor Temp 5 | K-Thermocouple | Ungrounded ⅛″ TC |
| 11 | RT6 | Reactor Temp 6 | K-Thermocouple | Ungrounded ⅛″ TC |
| 12 | RT7 | Reactor Temp 7 | K-Thermocouple | Ungrounded ⅛″ TC |
| 13 | RT8 | Reactor Temp 8 | K-Thermocouple | Ungrounded ⅛″ TC |

The e2V® IR-EK2 infrared sensor installed for on-line measurement of carbon dioxide and methane concentrations in the dry CAVoR reactor exhaust was monitored through a separate computer using software provided by the manufacturer. The detector was calibrated for carbon dioxide and closely tracked the periodic values obtained by gas chromatograph analysis. The methane analysis channel tracked relative concentrations and was useful for identifying the onset of gas release during heat up, but it was not precisely calibrated.

Separate temperature controllers were installed for the vaporizer shell temperature, upper reactor shell temperature, and lower reactor shell temperature. The vaporizer internal cartridge heater was controlled manually using a variable transformer and digital temperature readout for its internal thermocouple.

### Asteroid Simulant Preparation and Characterization

Based on the literature citations, various substrates and organic compounds were identified and evaluated for preparation of simulant for use during the initial Phase I CAVoR program. The goal was to create a simulant with the ability to bind water and to include organic matter of a composition and concentration reasonably representative of carbonaceous asteroids for the purpose of evaluating the CAVoR process.

### Inorganic Substrate

An inorganic clay substrate capable of holding water in the quantities needed to represent carbonaceous chondrite asteroid compositions (on the order of 15 percent water by weight) while having a granular and somewhat friable particle structure such as that described in the literature (O'Neill, 1979) was desired. Granular montmorillonite clay, which is described as a soft phyllosilicate mineral, was identified as a suitable substrate based on these desired characteristics. This material is capable of absorbing significant quantities of both hydrophobic and hydrophilic compounds. A commercial bulk absorbent product (Condor® 35UX85) was obtained for the initial Phase I work.

The particle size distribution of the as-received montmorillonite was determined by dry sieving. The following table summarizes the results for both the as-received material and the material after excluding the coarsest particles (greater than 8 mesh or 2.5 millimeters). The finer (less than 8 mesh fraction) was chosen for CAVoR testing to minimize potential gas channeling through the Phase I reactor and to facilitate recovery and separation from the activated carbon guard bed installed at the bottom of the reactor.

TABLE 4
Montmorillonite Clay Substrate Particle Size Analysis.

| | | | As-Received | | <8 Mesh Fraction | |
|---|---|---|---|---|---|---|
| | | | Wt % | Cumulative | Wt % | Cumulative |
| Particle Size | | | Retained | % Passing | Retained | % Passing |
| <4 | M | >5156 | 0.0 | 100.0 | 0 | |
| 4 × 8 | M | 2464 × 5156 | 41.6 | 58.4 | 0 | 100 |
| 8 × 12 | M | 1700 × 2464 | 38.8 | 19.6 | 66.4 | 33.6 |
| 12 × 20 | M | 850 × 1700 | 16.8 | 2.8 | 28.7 | 4.9 |
| 20 × 40 | M | 425 × 850 | 2.6 | 0.2 | 4.5 | 0.3 |
| <40 | M | <425 | 0.2 | | 0.3 | |

| | | |
|---|---|---|
| Total | 100.0 | 100.0 |

The particle size analysis shows that the bulk clay has a relatively narrow particle size range. About 80 percent of the as-received clay particles are between 4 and 8 mesh (5156 and 2464 microns). After removing the coarsest fraction (>8 mesh, or >2464 microns), about 95 percent of the particles are between 8 and 20 mesh (1700 and 850 microns). The sample contains well less than one percent of fine particles or dust. Such a distribution may not be entirely representative of material excavated from an asteroid, but the fairly uniform, granular structure facilitated initial Phase I experimentation with respect to handling and evaluation of chemical reactions. The bulk density of the as-received montmorillonite sample was measured at 0.64 kg/L and calculated to be about 0.73 kg/L when infused with water and organic matter as an asteroid simulant. Some of the candidate organic constituents (such as tar sands and oil shale) also contain inorganic fractions that would enhance the concentration of finer particles.

The as-received and <8 mesh particle size fractions of montmorillonite clay were subjected to thermogravimetric analysis to establish weight loss as a function of temperature. Samples were ramped up to a maximum of 800° C. in increments and weighed at each step. A typical heating rate of 8° per minute and a hold time of 30 minutes were used in a muffle furnace that allows the sample to be exposed to air.

Weight loss data from the as-received and <8 mesh fractions of clay were similar. The weight loss data suggest about one percent of free water (based on weight loss up to 200° C.). About 3 percent total weight loss was noted at 500° C. A total of 4.5 to 5.5 percent weight loss was noted at 800° C., possibly due to release of bound water, conversion of hydroxyl groups, or release of carbon dioxide from carbonates, if present. Reforming experiments were based on the assumption that the montmorillonite clay contains 4.5 percent moisture released in the temperature range of interest for the reforming reactions.

A sample of <8 mesh montmorillonite clay was infused with moisture to the target 15 percent total value used for CAVoR reforming experiments. The sample was sealed after moisture addition to allow equilibration of moisture within pores. This sample was subjected to a thermogravimetric analysis using methods similar to those applied to the as-received clay sample described above. The thermogravimetric analysis showed that about 80 percent of the total moisture was released readily below about 150° C., indicating its presence as unbound water. The remaining bound moisture was gradually released as the sample was heated to a maximum of 800° C.

Some additional weight changes related to mineral transformations may also have contributed to the characteristics described above. The montmorillonite exhibited a color change from grey to reddish-tan as it was heated. The color change, which occurred between 600 and 800° C., may be due to increasing the oxidation state of iron contained in the silicate structure. The montmorillonite did not exhibit any significant fracturing, dust formation, or fusion as a result of heating.

Further experimentation showed that the montmorillonite clay saturates at 50 weight percent water. Additional water added to the clay above that concentration was not retained within the pores. This result suggests that the montmorillonite could also easily absorb organic matter in addition to water if a liquid hydrocarbon were used for experimentation.

An example CAVoR reforming test feed was prepared to evaluate the use of clay as a substrate. Water was added to the as-received montmorillonite (already containing an estimated 4.5 percent moisture) to characterize the nature of the material when pre-loaded to an approximate 15 weight percent total moisture content. Assuming an as-received concentration of 4.5 weight percent moisture, additional water was added to bring the total to 15 weight percent with allowance for additional organic matter to make up a 200 gram feed sample containing 167.2 grams as-received montmorillonite, 22.8 grams added water, plus 10 grams of organic matter (resulting in a final 15 weight percent water and 5 percent organic matter with balance dry clay). The 22.8 grams of water was easily taken up by the clay. The surfaces initially darkened, but gradually returned to the as-received grey color as the water distributed through the interior of the clay particles. Experiments showed that the clay substrate with infused water could accommodate the addition of a wide variety of organic compound additions at the target concentrations.

### Organic Constituent Candidates

Several candidate organic materials were identified for use in preparing an asteroid simulant containing about five weight percent hydrocarbons in a matrix with about 15 percent water and balance phyllosilicate clay. Materials selected for evaluation were based on their compositions and characteristics relative to the kerogen-type material expected to be predominant in carbonaceous chondrite asteroids, the concentration and nature of inorganic matter in the organic candidate samples, the expected reactivity or release of the organic matter as temperature is increased, and the handling characteristics with respect to preparing repeatable, representative blends.

One candidate organic constituent for a CAVoR simulant is oil sands. Oil sand consists of quartz (silica) sand grains that are surrounded by a layer of water and clay, which is covered in a heavy oil, or bitumen (Oil Sands Discovery Centre, 2015). Greater than 12 percent bitumen content is considered a rich oils sands deposit. The inorganic matter consists mostly of angular, abrasive silica grains. The bitumen contained in the oil sands has a typical analysis of about 83 percent carbon, 10 percent hydrogen, 1 percent oxygen, 5 percent sulfur, and less than 1 percent nitrogen. A sample of oils sands was obtained for evaluation and testing from the Oil Sands Discovery Centre in Canada. The oil sands consist of agglomerated oily sand that it not particularly sticky. This material can be blended with clay to make a reasonably uniform simulant mixture. Literature along with testing at Pioneer were used to estimate the total organic content of the oil sands at 10.5 percent by mass with a water content of 5.5 percent.

A sample of Gilsonite® or “asphaltum” was obtained from American Gilsonite in the form of a black powder of <200 mesh (<74 microns) particle size. Gilsonite has a wide molecular weight distribution, contains very little inorganic matter, and exhibits softening at less than 200° C. (Burman and Romagosa, 1990). Gilsonite contains about 84 percent carbon, 10 percent hydrogen, 3 percent nitrogen, 1 percent oxygen, and less than 0.5 percent sulfur with very low ash (inorganic) content. This material can be dry-blended with clay containing water to constitute an asteroid simulant.

Texas crude oil was obtained as an organic material that can be infused into montmorillonite clay pores along with water to make an asteroid simulant. The sample obtained for potential testing was relatively low viscosity and was easily poured. Typical crude oil contains about 84 percent carbon, 14 percent hydrogen, 1-3 percent sulfur, 1 percent oxygen and nitrogen, and 0.1 percent minerals and salts (American Petroleum Institute, 2011).

Additional samples of bitumen extracted from oil sands (via hot water/steam) and coke (resulting from bitumen upgrading such as thermal cracking or hydroconversion) were also obtained for evaluation. The extracted bitumen is a very thick, sticky, tar-like substance that is difficult to handle at ambient temperature. Production of a uniform mixture of extracted bitumen and clay would be nearly impossible without significantly heating the bitumen and substrate while distributing the organic matter. The coke fraction consists mostly of a black powder with some agglomerates and chunks up to about ¼-inch (0.635-cm). This material could be dry-blended with clay containing water to constitute an asteroid simulant.

Another candidate from the organic chemical composition standpoint is terrestrial oil shale, which contains kerogen as its primary organic constituent. A well-characterized Colorado oil shale prepared as a geochemical reference sample was found to be available from the U.S. Geological Survey (USGS) in Lakewood, Colo. The USGS, who also has experience in the preparation of simulants for other ISRU applications, provided data for this sample (GRM ShPYR-1). The sample is generally described as being composed of kerogen, dolomite, quartz, and feldspars and is primarily meant for analytical quality control related to oil shale extraction. The projected oil and water yields for this shale are 12 percent and 1 percent, respectively, when subjected to pyrolysis conditions used for extraction of oil from shale. USGS provided an analysis of 13.7 weight percent organic carbon in the as-supplied shale, which along with estimated 1.25:1 H:C and 0.075:1 O:C molar ratios was used to calculate a composition of the oil shale organic matter at 83.0 percent carbon, 8.65 percent hydrogen, and 8.35 percent oxygen (by weight). Similar Colorado oil shales are reported to contain between 0.25 and 2 percent sulfur (Stanfield et al., 1951). This material contains a wide range of particle sizes from the maximum 2.5 millimeter (8 mesh) down to dust.

A nominal 200 gram CAVoR reactor test charge consisting of a mixture of roughly 61 grams of oil shale, 115 grams of montmorillonite clay, and 25 grams of added water would provide the target 15 percent total water and 5 percent organic matter composition.

Unlike the expected composition of carbonaceous chondrite asteroids, this particular oil shale contains a carbonate mineral (dolomite) that can release up to about 3 weight percent carbon dioxide upon heating. In contrast, carbonaceous chondrites have little apparent inorganic carbon content. In addition, the shale inorganic matter is likely much harder and less porous than asteroid phyllosilicate clays. Nevertheless, the mixture described was deemed to constitute a reasonable simulant for examination of the CAVoR process. The reference material available from USGS was supplied crushed to less than 8 mesh (similar to the clay sample). Due to costs for this reference material, it was used for initial Phase I CAVoR experiments after first scouting test conditions and procedures using the other organic feed materials described below.

The candidate organic samples described above were subjected to sequential heating for thermogravimetric analysis and evaluation of properties as a function of temperature. Samples were heated in 100° C. increments up to 800° C. to determine weight loss, physical properties, and residual ash content. A muffle furnace was used for the experiments, which were carried out in shallow ceramic dishes of about 4-inches (10.2-cm) diameter. The samples were exposed to air in the furnace (via natural convection only), which provides at least a relative indication of their potential behavior in the transient oxygenated environment in the steam reforming reactor.

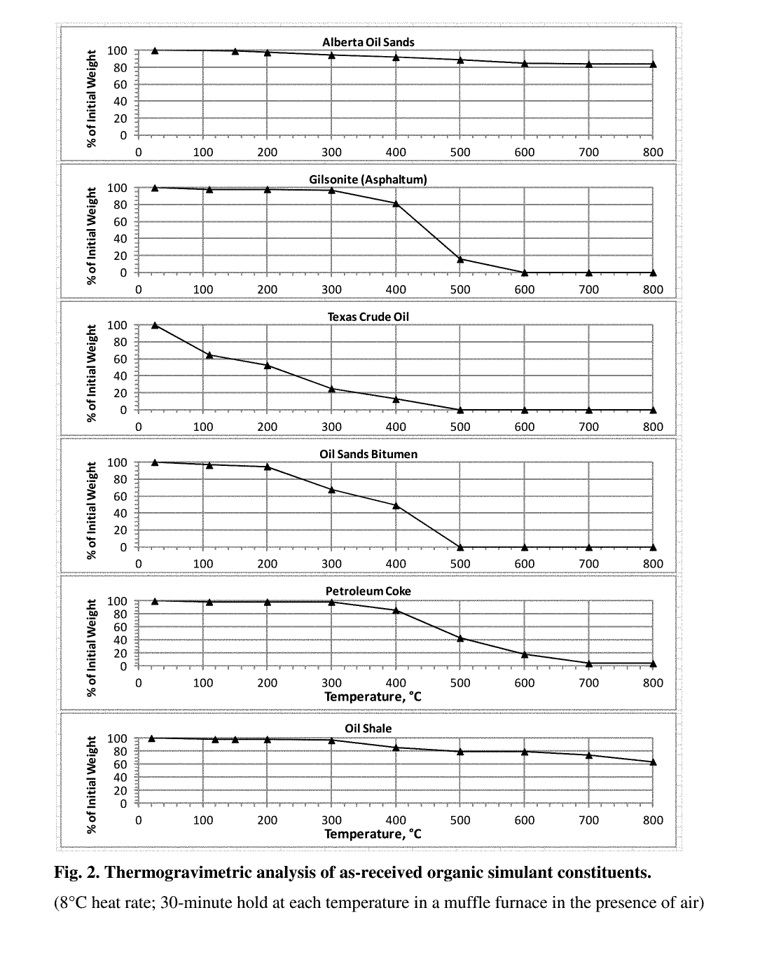

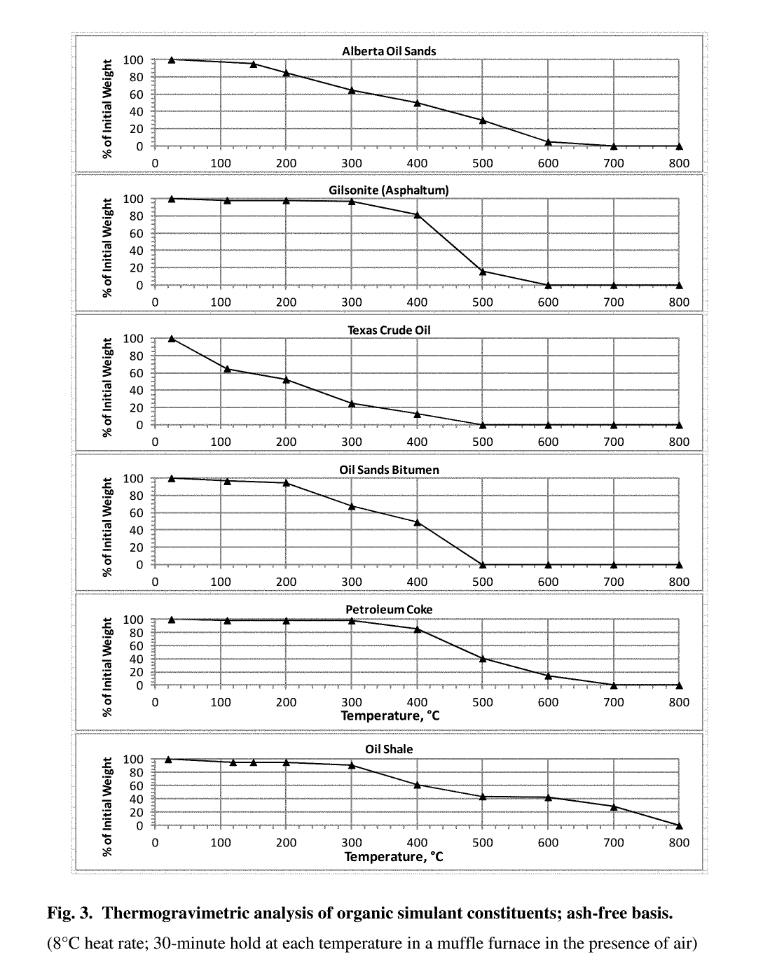

The loss of mass versus temperature is shown in FIG. 2 and FIG. 3 for each as-received material and for each material on an ash-free basis (hydrocarbon portion only), respectively. Data and observations made during the thermogravimetric analyses are summarized in the following tables.

TABLE 5
Thermogravimetric Analysis of Alberta Oil Sands.

| | As-Received | | Ash-Free Basis | | |
|---|---|---|---|---|---|
| Temp, | % of | % Wt | % of | % Wt | |
| ° C. | Starting Wt | Loss | Starting Wt | Loss | Comments |
| 25 | 100.0 | 0.0 | 100.0 | 0.0 | Black, slightly sticky |
| 150 | 99.2 | 0.8 | 95.0 | 5.0 | No significant change |
| 200 | 97.6 | 2.4 | 85.0 | 15.0 | Slight odor release, otherwise no |
| | | | | | signficant change |
| 300 | 94.4 | 5.6 | 65.0 | 35.0 | Sticky, more coherent, black |
| 400 | 92.1 | 7.9 | 50.0 | 50.0 | Harder agglomerates, not sticky |
| 500 | 88.9 | 11.1 | 30.0 | 70.0 | Mostly grey powder, some remaining |
| | | | | | black organic matter |
| 600 | 84.9 | 15.1 | 5.0 | 95.0 | Lighter grey, free flowing |
| 700 | 84.1 | 15.9 | 0.0 | 100.0 | Light grey, a few reddish pieces |
| 800 | 84.1 | 15.9 | 0.0 | 100.0 | Light grey and tan ash (84.1% ash) |

TABLE 6
Thermogravimetric Analysis of Gilsonite.

| | As-Received | | Ash-Free Basis | | |
|---|---|---|---|---|---|
| Temp, | % of | % Wt | % of | % Wt | |
| ° C. | Starting Wt | Loss | Starting Wt | Loss | Comments |
| 25 | 100.0 | 0.0 | 100.0 | 0.0 | Black powder |
| 110 | 98.1 | 1.9 | 98.1 | 1.9 | |
| 200 | 98.1 | 1.9 | 98.1 | 1.9 | Slightly melted on top |
| 300 | 97.2 | 2.8 | 97.2 | 2.8 | Glazed on top |
| 400 | 81.5 | 18.5 | 81.5 | 18.5 | More melting/off-gassing pockets |
| 500 | 15.7 | 84.3 | 15.7 | 84.3 | Mostly cohesive with some fines |
| 600 | 0.0 | 100.0 | 0.0 | 100.0 | Mostly dark reddish/tan powdered ash |
| 700 | 0.0 | 100.0 | 0.0 | 100.0 | |
| 800 | 0.0 | 100.0 | 0.0 | 100.0 | (<0.7% ash) |

TABLE 7
Thermogravimetric Analysis of Texas Crude Oil.

| | As-Received | | Ash-Free Basis | | |
|---|---|---|---|---|---|
| Temp, | % of | % Wt | % of | % Wt | |
| ° C. | Starting Wt | Loss | Starting Wt | Loss | Comments |
| 25 | 100.0 | 0.0 | 100.0 | 0.0 | |
| 110 | 65.0 | 35.0 | 65.0 | 35.0 | |
| 200 | 52.5 | 47.5 | 52.5 | 47.5 | Very fluid hot, solidified at ambient temperature |
| 300 | 25.0 | 75.0 | 25.0 | 75.0 | Black solid shiny surface, most odor gone |
| 400 | 12.5 | 87.5 | 12.5 | 87.5 | Black/brown surface |
| 500 | 0.0 | 100.0 | 0.0 | 100.0 | |
| 600 | 0.0 | 100.0 | 0.0 | 100.0 | Mostly ash |
| 700 | 0.0 | 100.0 | 0.0 | 100.0 | |
| 800 | 0.0 | 100.0 | 0.0 | 100.0 | (<1% ash) |

TABLE 8
Thermogravimetric Analysis of Oil Sands Bitumen.

| | As-Received | | Ash-Free Basis | | |
|---|---|---|---|---|---|
| Temp, | % of | % Wt | % of | % Wt | |
| ° C. | Starting Wt | Loss | Starting Wt | Loss | Comments |
| 25 | 100.0 | 0.0 | 100.0 | 0.0 | Very viscous/tar like |
| 110 | 96.6 | 3.4 | 96.6 | 3.4 | Viscous black liquid |
| 200 | 94.9 | 5.1 | 94.9 | 5.1 | Fluid black liquid |
| 300 | 67.8 | 32.2 | 67.8 | 32.2 | Shiny black surface |
| 400 | 49.2 | 50.8 | 49.2 | 50.8 | Light crust on surface |
| | | | | | with voids below |
| 500 | 0.0 | 100.0 | 0.0 | 100.0 | Mostly ash |
| 600 | 0.0 | 100.0 | 0.0 | 100.0 | |
| 700 | 0.0 | 100.0 | 0.0 | 100.0 | |
| 800 | 0.0 | 100.0 | 0.0 | 100.0 | (<0.9% ash) |

TABLE 9
Thermogravimetric Analysis of Alberta Oil Sands Petroleum Coke.

| | As-Received | | Ash-Free Basis | | |
|---|---|---|---|---|---|
| Temp, | % of | % Wt | % of | % Wt | |
| ° C. | Starting Wt | Loss | Starting Wt | Loss | Comments |
| 25 | 100.0 | 0.0 | 100.0 | 0.0 | |
| 110 | 98.6 | 1.4 | 98.6 | 1.4 | |
| 200 | 98.6 | 1.4 | 98.6 | 1.4 | No significant change |
| 300 | 98.6 | 1.4 | 98.6 | 1.4 | |
| 400 | 86.1 | 13.9 | 85.5 | 14.5 | Slight ashing |
| 500 | 43.1 | 56.9 | 40.6 | 59.4 | Mostly ash |
| 600 | 18.1 | 81.9 | 14.5 | 85.5 | Mostly light reddish/tan powdered ash |
| 700 | 4.2 | 95.8 | 0.0 | 100.0 | |
| 800 | 4.2 | 95.8 | 0.0 | 100.0 | (4.2% ash) |

TABLE 10
Thermogravimetric Analysis of Colorado Oil Shale.

| | As-Received | | Ash-Free Basis | | |
|---|---|---|---|---|---|
| Temp, | % of | % Wt | % of | % Wt | |
| ° C. | Starting Wt | Loss | Starting Wt | Loss | Comments |
| 20 | 100.00 | 0.00 | 100.00 | 0.00 | Grey/tan |
| 119 | 98.4 | 1.6 | 95.6 | 4.4 | |
| 150 | 98.4 | 1.6 | 95.6 | 4.4 | |
| 200 | 98.4 | 1.6 | 95.6 | 4.4 | |
| 300 | 96.8 | 3.2 | 91.1 | 8.9 | Dark tan |
| 400 | 86.2 | 13.8 | 61.7 | 38.3 | Brown black/grey |
| 500 | 79.8 | 20.2 | 43.9 | 56.1 | |
| 600 | 79.4 | 20.6 | 42.8 | 57.2 | |
| 700 | 74.2 | 25.8 | 28.3 | 71.7 | |
| 800 | 64.0 | 36.0 | 0.0 | 100.0 | Pink/tan |

The results in the thermogravimetric analysis figures and tables show some significant differences in weight loss as a function of temperature. The small weight loss at temperatures below 200° C. for the Alberta oils sands probably represents moisture loss from the as-received sample. In contrast, the lower-temperature weight loss from the Texas crude oil sample probably represents loss of light hydrocarbon components.

The oil sands and petroleum coke samples were the most refractory in that higher temperatures were required to oxidize the last of the organic matter. The Texas crude oil and extracted bitumen exhibited the highest weight loss at lower temperatures. The extracted bitumen (presumably using hot water/steam) may contain more lighter fractions than as-received oil sands, or the high mass of inorganic matter in the oil sands may have inhibited release of organic matter compared to the extracted sample. Comments in the table above indicate temperatures at which significant physical changes to samples took place.

The Gilsonite and petroleum coke samples are dry, generally free-flowing powders or granules. The Texas crude oil sample readily spread across the shallow dish while the extracted bitumen sample remained as a viscous blob in the center of the ceramic dish.

Only the petroleum coke contained significant ash (about 4 percent of the starting mass). The ash mass of the other samples was very low (generally less than about 1 percent). However, the remaining inorganic matter was generally of very low density and appeared relatively high in volume compared to the starting samples. Only the Texas crude oil ash showed some signs of fusion after being calcined at 800° C.

Surface melting of Gilsonite was evident at 200° C., partial combustion was noted at 500° C., and the sample was almost entirely burned at 600° C.

After heating Colorado oil shale to 400° C., the contained organic matter was observed on the mineral surfaces. The fully calcined 800° C. residue was similar in appearance to the feed despite burnout of organic matter.

In addition to the major simulant constituents described above, additional minor constituents can be included to enhance the fidelity of the asteroid simulant. For example, a sample of granular magnetite (F.J. Brodman & Co.; 60×100 mesh; 99.8% Fe₃O₄) was procured for testing. Magnetite is an expected potentially significant component of carbonaceous chondrite asteroids and is a potential source for additional oxygen recovery via reduction by hydrogen or carbon monoxide during reforming. By comparing the mineralogy of magnetite before and after processing, the overall reduction-oxidation characteristics that occur as the autothermal reforming reaction front moves along the length of the reactor can be determined. This is helpful in determining whether oxygen contained in magnetite (or other minerals) can be obtained directly during reforming (via reduction to lower oxides or metals) or whether the processed CAVoR residue would be a suitable candidate for a separate hydrogen reduction step to recover oxygen from iron oxides in the form of water.

In summary, the results of characterizations of both the montmorillonite clay substrate and candidate organic materials showed that a number of potentially suitable carbonaceous chondrite asteroid simulant formulations can be prepared for proof-of-concept testing of the Carbonaceous Asteroid Volatile Recovery System. Experiments were conducted using a combination of clay along with oil sands, gilsonite, petroleum coke, and oil shale with moisture content adjusted to provide a fixed 15 percent total moisture and 5 percent organic matter. The physical and chemical characteristics of Gilsonite appear to be reasonably well matched to the projected characteristics of organic matter in carbonaceous chondrite asteroids, and Gilsonite was used for much of the experimental program. The responses of kerogen in oil shale as well as the organic matter in oil sands and petroleum coke were also evaluated during the experimental program.

### CAVoR Reforming Experiments

Experiments carried out during the initial CAVoR Phase I program demonstrated the feasibility of using autothermal steam reforming for the recovery of water and organic matter from a variety of carbonaceous asteroid simulant compositions to produce syngas and water, and in some cases condensable organic matter. With the addition of a downstream methanation/electrolysis unit, the dry CAVoR product gas (consisting primarily of hydrogen, carbon monoxide, and carbon dioxide) can be converted to methane and oxygen propellant constituents as demonstrated by Pioneer during other work (Carrera et al., 2013).

The effects of the composition of the organic component of the feed as well as key process parameters including processing rate, temperature, oxygen:carbon ratio, and steam:carbon ratio were established from the experimental results. Shortcomings of the Phase I batch processing method were identified; however, evaluation of the test results led to a conceptual continuous processing design that is capable of achieving high heat recovery from the spent process material, which leads to reduced thermal and electrical heat inputs while maximizing hydrogen production during reforming. Continuous (or at least long-duration operation between shutdowns) also minimizes the production of heavier hydrocarbons that are evolved by pyrolysis as the system is preheated. The CAVoR Phase I experimental program approach and results are summarized below.

### System Operating Procedures and Shakedown Testing

The following describes the general procedures applied to CAVoR reforming experiments. A start up, operating, and shut down checklist was prepared prior to each experiment. The checklist procedures were revised as testing progressed during the initial Phase I program.

Each experiment was carried out by first adding a layer of >8 mesh (>2.5 mm) activated carbon pellets to serve as a guard against any oxygen bypassing the organic matter in the asteroid simulant. The activated carbon was found to be suitable for the application by separate testing to verify reaction with oxygen at high temperatures (ashing was observed at temperatures above about 500° C. in the presence of oxygen). The carbon was also found to absorb no water, CO₂, or hydrocarbons under the conditions of use, thereby making it suitable as a guard bed without interfering with test measurements. The carbon was pre-dried at a temperature of about 150° C. prior to use. An amount of about 30 grams was used to fill the lower section of the reactor to just above the lowest of the eight internal reactor thermocouples (RT8).. A temperature rise of RT8 would indicate reaction of oxygen with the guard bed carbon. The activated carbon bulk density was only about 0.47 kg/L, about one-half that of typical asteroid simulants. As discussed later, the coarse carbon was easily separated from the finer reforming residue by sieving after each test. The mass of activated carbon recovered after each experiment was nearly the same as the feed mass. Carbon pellets spot checked from selected experiments were heated after use and were found to release no significant weight.

Next, the reactor was filled with asteroid simulant through the upper port. Depending on the asteroid simulant characteristics, a mass between about 170 and 200 grams was used to fill to a level just below the steam/oxygen injection port. The uppermost of the eight internal reactor thermocouples (RT1) marks the approximate upper sample level.

A typical simulant was prepared to contain <8 mesh montmorillonite clay, 15 weight percent total moisture concentration, and 5 percent organic matter. As an example, a 200 gram Gilsonite-containing simulant was prepared using 169.1 grams of <8 mesh montmorillonite clay (at an as-received moisture content of 4.5 percent). Water was uniformly sprayed onto the surfaces of the clay in an amount of 20.9 grams in a large ceramic dish. Gilsonite (<200 mesh or <74 microns) was blended into the wetted clay in an amount of 10 grams. The mixture was further blended and placed into a sealed jar to allow the moisture to equilibrate prior to loading into the reactor. Even the relatively low 5 percent concentration of organic matter rendered the overall mixture a very dark color compared to the light tan appearance of pure montmorillonite clay.

After loading simulant, a flow of helium was initiated to purge oxygen from the system after which the system was pressurized and checked for leaks. Instruments and analysis calibrations were also checked as part of the start-up procedure. In the Phase I batch apparatus, helium prevented product gases from diffusing back toward the oxygen/steam inlet. In addition, helium served as a tracer gas to precisely calculate dry exhaust gas flows for material balance calculations using the known helium feed rate (typically 90 to 100 sccm) and the analysis of helium in the exhaust. All experiments were carried out at near-ambient pressure (about 0.8 bar absolute) with only occasional application of system back pressure to withdraw samples from the condenser.

A protocol for preheating of the vaporizer (typically to about 700° C. in the cartridge heater) and reactor system (between 600 and 750° C. upper and lower reactor shell temperatures) was followed. When the reactor internal temperature achieved a specified target temperature (400 to 450° C.), steam and oxygen flows were started. Steam and oxygen rates were pre-set to provide a range of oxygen:carbon molar ratios (between 0.25:1 and 0.5:1) and steam:carbon molar ratios (between 1:1 and 2:1). These molar ratios were determined from the estimated mass of carbon loaded into the reactor as simulant (typically about 7 to 8 grams of contained carbon in 160 to 200 grams of simulant). The delivery rates of the calculated steam and oxygen masses were based on the target reaction time (from 40 to 60 minutes).

The progress of the reaction was readily apparent by the temperature rise associated with the partial oxidation exotherm as it proceeded down the length of the reactor.

When the reaction front approached the carbon guard bed at the bottom of the reactor, oxygen and steam flows were stopped and heaters were turned off while cooling down under a flow of helium. The oxygen and steam flows were stopped when the temperature in the second thermocouple from the bottom of the reactor (RT7) had peaked in temperature and had just begun to fall. In almost all cases, residue collected from the zone below this point appeared similar to the feed simulant. Residue collected from the zone above this point appeared to be completely free of organic matter.

A pneumatic system using a venturi powered by compressed air was used to collect residue from the reactor by vacuuming from the reactor and transporting to a collection receiver for weighing and analysis. A wand attached to the venturi pulls sample from the reactor through the transfer tubing to a receiver made from a sealed pail containing an outlet to which a filter felt was installed to collect and retain virtually all particles.

Except for the initial CAVoR reforming experiment, the vacuum wand was used to collect residue in two portions. The first portion included sample from the RT7 thermocouple level and above, which consists of material exposed to oxygen and steam during reforming. All of this material was finer than 8 mesh (2.5 mm) in size and in nearly all cases was virtually free of carbonaceous material. The remaining residue was then removed down to the support screen at the bottom of the reactor. The material in the lower section was sieved at 8 mesh to separate the carbon guard bed pellets from the asteroid simulant residue. In nearly each case, the <8 mesh fraction of residue from the lower section appeared similar to the feed simulant (i.e. unreacted organic matter was clearly visible). Weights of each residue fraction and of the carbon bed were recorded for material balance purposes. The pneumatic transport of residue allowed for repeated operation of the reactor system by removing just one sealed port after each experiment. The reactor remained mounted to the test stand along with all heaters, insulation, and instrumentation at all times during the test program. Only a short section of removable insulation was required to be handled to access the fill/discharge port.

Gas samples were taken during the course of each experiment at typical intervals of 5 to 7 minutes to track the product gas composition. Gases including helium, hydrogen, carbon monoxide, carbon dioxide, and methane were routinely analyzed by a Varian (Agilent) CP-4900® micro gas chromatograph using Galaxie® data acquisition software. An SRI Sulfur-in-Natural-Gas gas chromatograph equipped with a flame photometric detector for sulfur compounds and a flame ionization detector for hydrocarbons was used to analyze the dry product gas before and after a zinc oxide sulfur trap. PeakSimple® data acquisition software was used to interpret the SRI GC results. Hydrogen sulfide and carbonyl sulfide were detected and quantified in the CAVoR exhaust gases using the SRI GC. Small amounts of hydrocarbons other than methane were detected by gas chromatography, generally during sample preheating period just before steam and oxygen injection was started.

In addition to gas samples, condensate produced during each experiment was periodically discharged. Weights of the condensate were obtained to track the nature of moisture evolution from the asteroid simulants. Most of the water added to the montmorillonite clay substrate constituent of the asteroid simulants was released as the sample was preheated in the batch reactor. In most cases, a small amount of organic matter was evolved during the preheat portion of each experiment just before steam and oxygen injection was started.

The release of un-reformed organic matter during preheating was a shortcoming of the initial Phase I batch reactor system. However, from this observation along with analysis of additional experimental data, an improved method for processing asteroid regolith in a continuous flow reactor was devised. The continuous flow system, which ensures that all organic matter released from asteroid regolith passes through the hottest zone containing steam and oxygen, is detailed separately.

### Parametric CAVoR Phase I System Experiments

During the course of the CAVoR Phase I program, steam reforming experiments were successfully carried out using asteroid simulants prepared from four different organic constituents to evaluate the effect of the organic composition on the production of syngas for downstream methanation. In addition, experiments were carried out to evaluate the effect of reaction time as well as steam:carbon ratio and oxygen:carbon ratio on the reforming gas composition. Additional testing was conducted to determine the effects of the batch reforming reaction conditions on the oxidation state of magnetite, a potentially significant constituent of carbonaceous chondrite asteroids and a potential source of oxygen. The release of sulfur gas compounds and their capture on a sorbent were also characterized. Additionally, limited experimentation was carried out to determine whether supplemental oxygen could be injected in the lower part of the Phase I batch reactor to decompose organic matter released as pyrolysis gases during system heat up.

The CAVoR reactor system experiments demonstrated that steam reforming augmented by partial oxidation is a viable method for the recovery of organic matter and moisture from a wide variety of materials representing carbonaceous chondrite asteroid materials. A range of asteroid simulant compositions was successfully processed to generate syngas (suitable as feed to methanation) and water (which can in part be electrolyzed to produce oxygen) for the in-space manufacture rocket propellant. The CAVoR experimental results, in combination with modeling described later, led to identification of a continuous process system for production of syngas containing significant quantities of hydrogen via thermochemical reforming. Such a system can provide substantial electrical power savings by reducing electrolysis loads.

Potential shortcomings of the Phase I batch processing system were identified and evaluated to make design improvements to reduce or eliminate issues related to the release of organic compounds by pyrolysis during preheating of the batch system during start up. The resulting continuous (or long-duration operation) CAVoR reformer design is capable of achieving high levels of heat recovery from spent regolith, which is important given the large mass of inert or inorganic material that must be processed to recover the valuable water and organic compounds contained in carbonaceous asteroid materials.

Table 12 summarizes the experimental matrix for key CAVoR Phase I experiments. The table highlights the feed simulant compositions, reformer process conditions, and exhaust gas composition results. The mass accountability ranged for 88 to 103 percent throughout the test program.

TABLE 11
CAVoR Phase I Experimental Summary.

| | | | | | | | | | Target | |
|---|---|---|---|---|---|---|---|---|---|---|
| | | | | | | | | | Temperatures, | |
| | | Simulant | | | | | | | ° C. | |
| | | Feed | Target Conditions | | | Target Feed Rates | | | Vaporizer | |
| Run | Carbonaceous Asteroid | Mass, | | | Time, | He, | O₂, | H₂O, | Cartridge | Vaporizer |
| # | Simulant Composition | g | O₂:C | H₂O:C | min | sccm | sccm | ml/min | Heater | Shell |
| R1 | <8M Clay, 15% H₂O, 5% Organics | 161.5 | 0.5:1 | 1:1 | 60 | 100 | 106 | 0.17 | 700 | 600 |
| | (Gilsonite) | | | | | | | | | |
| R2 | <8M Clay, 15% H₂O, 5% Organics | 170.6 | 0.5:1 | 1:1 | 40 | 100 | 167 | 0.27 | 750 | 600 |
| | (Gilsonite) | | | | | | | | | |
| R3 | <8M Clay, 15% H₂O, 5% Organics | 164.5 | 0.5:1 | 1:1 | 40 | 100 | 161 | 0.26 | 750 | 600 |
| | (Gilsonite) | | | | | | | | | |
| R4 | <8M Clay, 15% H₂O, 5% Organics | 165.8 | 0.5:1 | 2:1 | 40 | 100 | 163 | 0.52 | 750 | 600 |
| | (Gilsonite) | | | | | | | | | |
| R5 | <8M Clay, 15% H₂O, 5% Organics | 168.9 | 0.5:1 | 1:1 | 20 | 100 | 331 | 0.53 | 720 | 600 |
| | (Gilsonite) | | | | | | | | | |
| R6 | <8M Clay, 15% H₂O, 5% Organics | 193.4 | 0.5:1 | 1:1 | 40 | 100 | 187 | 0.30 | 750 | 600 |
| | (Oil Sands) | | | | | | | | | |
| R7 | <8M Clay, 15% H₂O, 5% Organics | 164.6 | 0.5:1 | 1:1 | 40 | 100 | 173 | 0.28 | 750 | 600 |
| | (Petroleum Coke) | | | | | | | | | |
| R8 | <8M Clay, 15% H₂O, 5% Organics | 164.7 | 0.5:1 | 1:1 | 40 | 100 | 162 | 0.26 | 750 | 600 |
| | (Gilsonite) | | | | | | | | | |
| R9 | <8M Clay, 15% H₂O, 5% Organics | 160.7 | 0.5:1 | 1:1 | 40 | 100 | 158 | 0.25 | 750 | 600 |
| | (Gilsonite) | | | | | | | | | |
| R10 | <8M Clay, 15% H₂O, 5% Organics | 154.8 | 0.5:1 | 2:1 | 40 | 100 | 152 | 0.49 | 750 | 600 |
| | (Gilsonite) | | | | | | | | | |
| R11 | <8M Clay, 15% H₂O, 5% Organics | 199.9 | 0.5:1 | 1:1 | 40 | 100 | 194 | 0.31 | 725 | 600 |
| | (Oil Shale) | | | | | | | | | |
| R12 | <8M Clay, 15% H₂O, 5% Organics | 197.5 | 0.5:1 | 2:1 | 40 | 100 | 191 | 0.62 | 725 | 600 |
| | (Oil Shale/Magnetite) | | | | | | | | | |
| R13 | <8M Clay, 15% H₂O, 5% Organics | 194.5 | 0.5:1 | 2:1 | 40 | 100 | 189 | 0.61 | 725 | 600 |
| | (Oil Shale/Supplemental O₂) | | | | | | | | | |
| R14 | <8M Clay, 15% H₂O, | 173.6 | 0.25:1 | 2:1 | 40 | 100 | 85 | 0.55 | 725 | 600 |
| | 5% Organics (Gilsonite) | | | | | | | | | |

| | Target | | | | | | | | | | | |
|---|---|---|---|---|---|---|---|---|---|---|---|---|
| | Temperatures, | | Average He-Free | | | | | | | | | Mass |
| | ° C. | | Dry Exhaust Gas Composition, | | | | Gas Production, | | | | | Balance |
| Run | Upper Reactor | Lower Reactor | volume % | | | | grams | | | | | Closure, |
| # | Shell | Shell | H₂ | CO | CH₄ | CO₂ | H₂ | CO | CH₄ | CO₂ | Total | % |
| R1 | 400 | 400 | 21.6 | 15.4 | 5.0 | 57.9 | 0.15 | 1.49 | 0.28 | 8.76 | 10.68 | 98.7 |
| R2 | 400 | 400 | 6.4 | 23.1 | 3.0 | 67.5 | 0.05 | 2.38 | 0.18 | 10.97 | 13.58 | 93.8 |
| R3 | 600 | 600 | 19.5 | 25.6 | 5.7 | 49.3 | 0.15 | 2.81 | 0.36 | 8.51 | 11.82 | 101.7 |
| R4 | 600 | 600 | 17.2 | 21.1 | 6.3 | 55.4 | 0.13 | 2.16 | 0.37 | 8.91 | 11.57 | 97.1 |
| R5 | 600 | 600 | 15.8 | 19.3 | 7.0 | 57.9 | 0.11 | 1.84 | 0.38 | 8.67 | 11.00 | 87.8 |
| R6 | 600 | 600 | 19.7 | 14.7 | 7.8 | 57.8 | 0.15 | 1.62 | 0.49 | 9.99 | 12.26 | 98.7 |
| R7 | 600 | 600 | 3.6 | 9.5 | 3.7 | 83.2 | 0.03 | 1.07 | 0.24 | 14.78 | 16.12 | 93.5 |
| R8 | 700 | 700 | 22.4 | 14.3 | 7.3 | 56.0 | 0.20 | 1.76 | 0.51 | 10.84 | 13.31 | 97.7 |
| R9 | 650 | 650 | 17.5 | 15.4 | 6.1 | 61.1 | 0.14 | 1.77 | 0.40 | 11.04 | 13.35 | 98.3 |
| R10 | 650 | 650 | 18.7 | 17.9 | 5.5 | 57.9 | 0.15 | 1.97 | 0.35 | 9.99 | 12.45 | 98.5 |
| R11 | 600 | 600 | 9.9 | 7.7 | 6.1 | 76.3 | 0.10 | 1.10 | 0.50 | 17.17 | 18.87 | 94.9 |
| R12 | 700 | 700 | 18.8 | 7.0 | 4.2 | 70.0 | 0.25 | 1.31 | 0.45 | 20.40 | 22.41 | 96.2 |
| R13 | 700 | 700 | 17.2 | 6.9 | 6.4 | 69.4 | 0.23 | 1.28 | 0.68 | 20.17 | 22.36 | 95.1 |
| R14 | 750 | 750 | 37.7 | 12.5 | 6.9 | 42.9 | 0.45 | 2.08 | 0.66 | 11.24 | 14.42 | 98.5 |

### Effect of Organic Matter Composition

The montmorillonite clay described in the Asteroid Simulant Preparation section was used as the substrate into which moisture was infused and with which the organic constituents were blended in all four types of simulant prepared and tested during Phase I. The four different types of organic matter varied considerably in physical and chemical characteristics, which was useful for evaluation of CAVoR over a range of feeds. For example, Gilsonite contained very little inorganic material and was therefore mixed into simulant blends as a fine powder containing pure organic matter (resulting in the great majority of the feed simulant consisting of clay). Petroleum coke was a hard carbon containing a range of particle sizes up to the 8 mesh maximum size in simulant. The measured 4 percent inorganic matter in petroleum coke was accounted for during simulant preparation, but like Gilsonite, the majority of inorganic material in the simulant consisted of montmorillonite clay. The oil sands sample contained only about 10.5 by weight of total organic content. Therefore, the relative amount of clay was reduced significantly while the mass of sticky oil sands, including its contained inorganic material, was increased significantly in the simulant compared to the high-organic-content components of the two simulants described above. The oil shale sample analysis by the U.S.G.S. indicated a total organic (C+H+O) content of about 16.5 percent by weight. Therefore, like the oil sands, the total oil shale sample mass was considerably greater in proportion to the clay in the high-organic feeds. However, the oil shale residue appearance was similar in color and overall characteristics to the as-received material (and to the montmorillonite clay substrate). The organic matter is apparently contained within tight pores that release the organic matter during reforming. The oil shale did contain significantly more fine particles than the clay substrate.

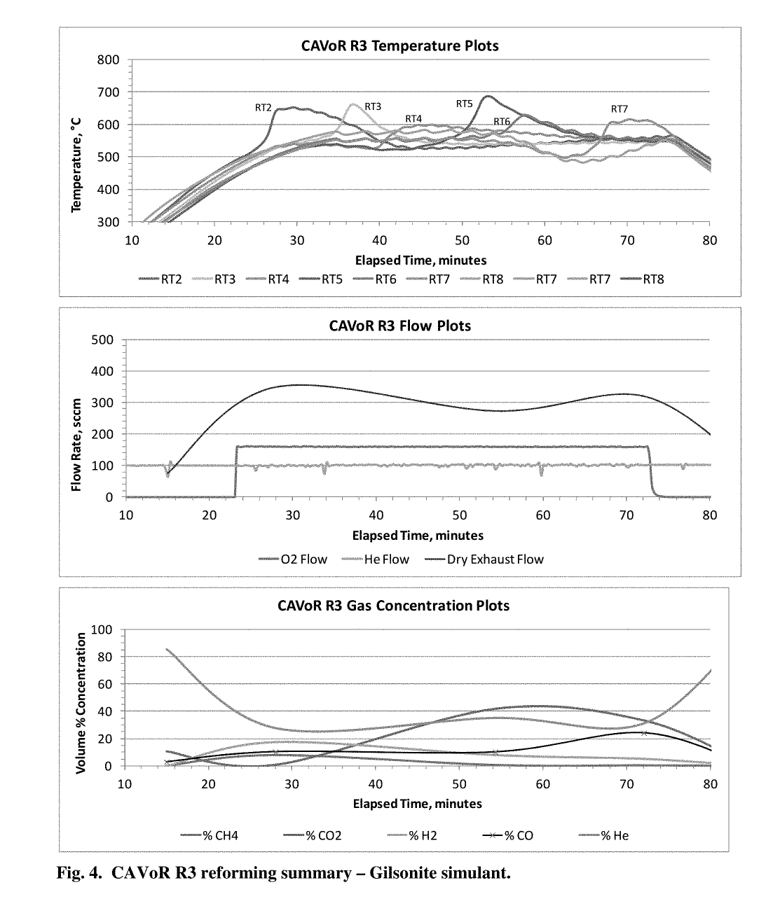

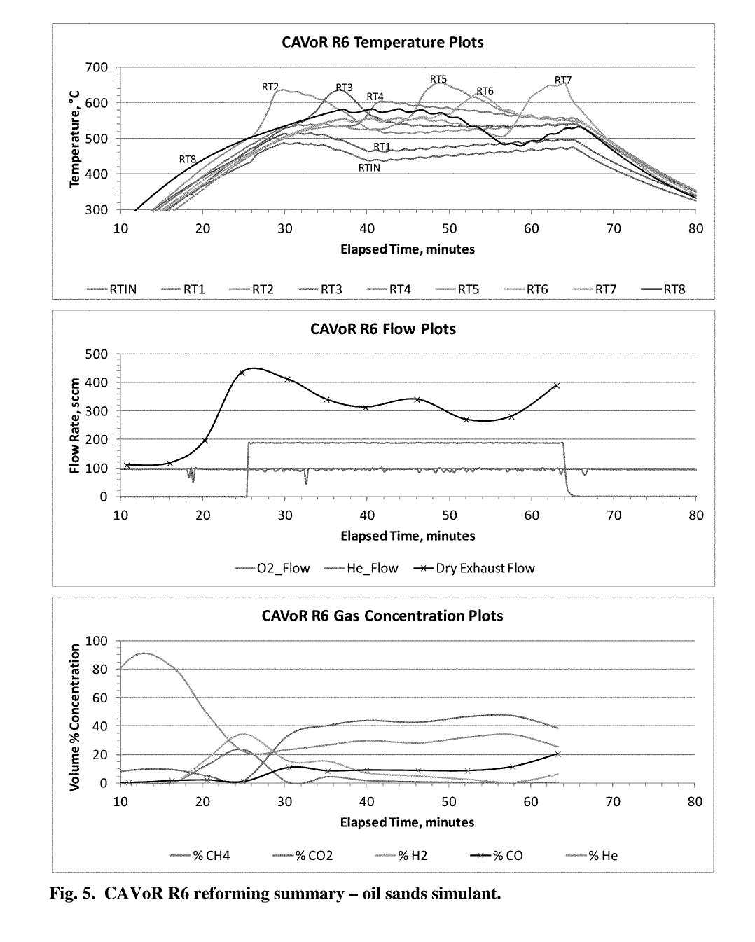

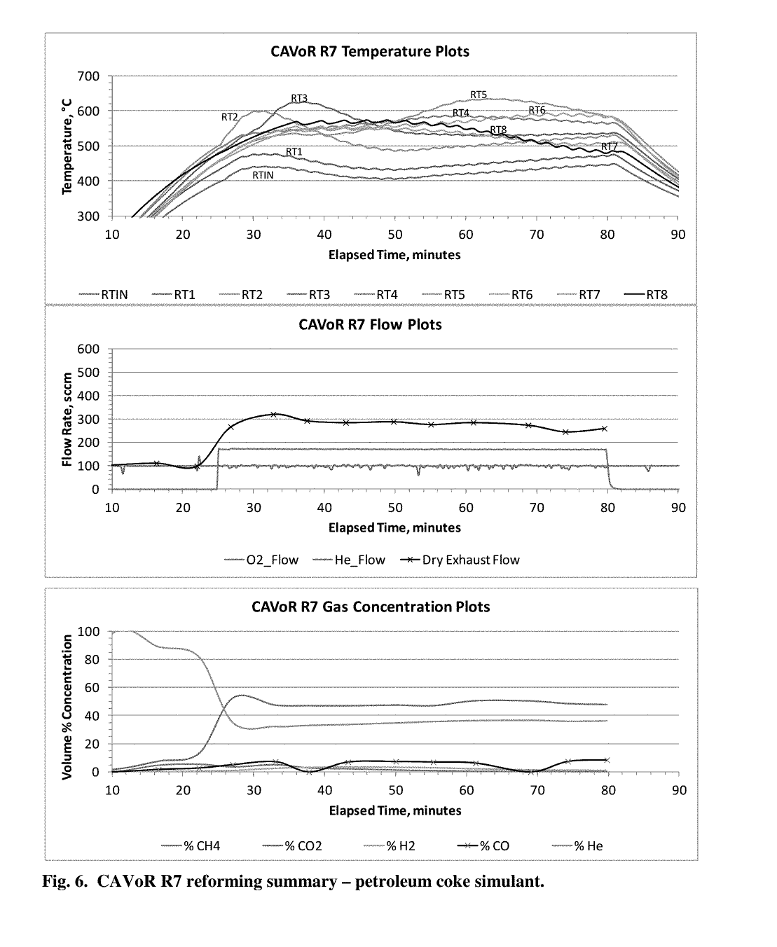

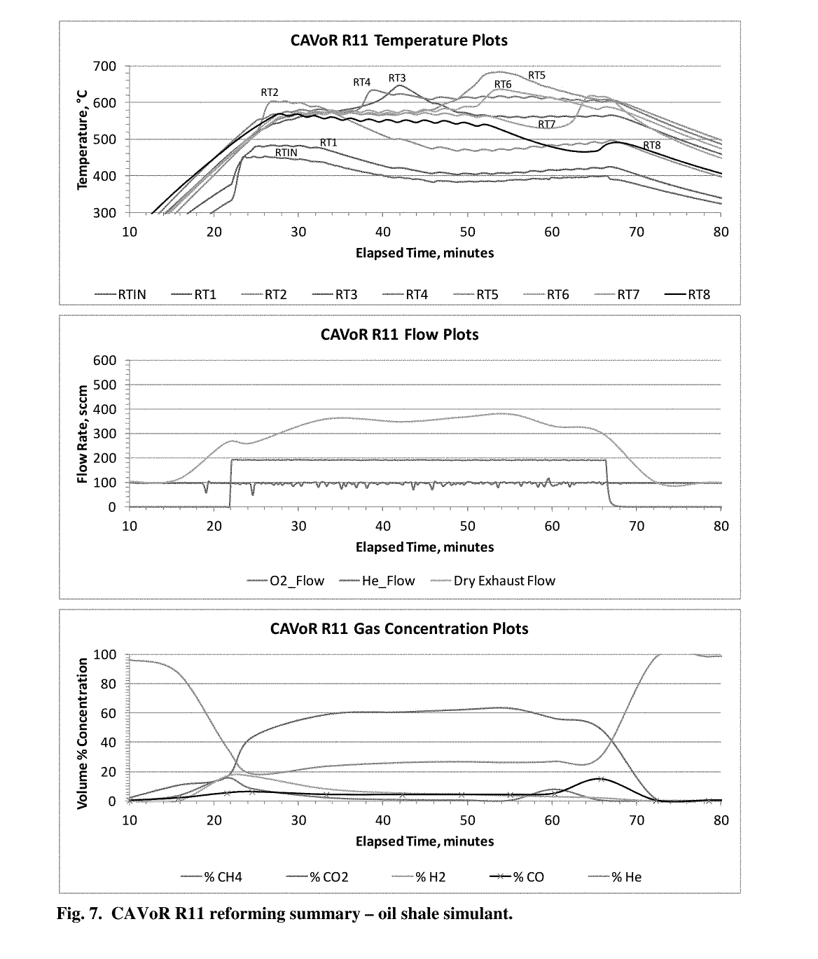

Each of the feeds was processed at a nominal 0.5:1 oxygen:carbon ratio and 1:1 steam:carbon ratio during steam reforming for a target 40 minute reaction time in the Phase I batch reactor using a 600° C. set point on the reactor shell temperatures. FIG. 4 through FIG. 7 show the temperature, gas flow, and gas composition profiles for each of the asteroid feed simulants.

The temperature, gas flow rate, and gas concentration plots for all four simulant types were similar. Each simulant exhibited a characteristic temperature rise as the partial oxidation/reforming reaction front moved downward past the internal reactor thermocouples. In the temperature plots, thermocouples RT2 through RT7 were in the batch testing reaction zones (RT1 was just above the simulant bed; RT8 was in the carbon pellet guard bed just below the simulant). In some cases, the reaction exotherms (as indicated by temperature rises at each thermocouple location) did not take place at precisely uniform time intervals. This is thought to be a result of some sample non-uniformity or oxygen channeling in the simulant bed. However, the actual reaction times were close to the target value of 40 minutes, indicating that the estimated feed compositions were reasonably accurate, and nearly complete removal of organic matter occurred within the reaction zone for each simulant. The petroleum coke sample exhibited fewer noticeable exotherms than the other samples, possibly indicating the more-refractory nature of this sample (which is the residue remaining after all lighter hydrocarbons are removed by pyrolysis at high temperature).

The helium sweep gas and oxygen flow rates were steady during each experiment. The larger downward spikes evident in the helium gas flow resulted from brief flow reductions that occurred as the reactor exhaust valve was closed to build slight pressure to discharge condensate samples, resulting in a reduction of helium flow (which was regulated at a feed pressure just above ambient). A spike in exhaust gas flow rates began immediately after starting steam and oxygen injection. The dry exhaust gas flow rate remained fairly constant during the steam and oxygen injection periods.

During periods when steam and oxygen injection were on, the helium sweep gas became a minority constituent in the dry exhaust gas. The dry reformer exhaust gas compositions were similar during each experiment except for that of the petroleum coke simulant. Carbon dioxide was the predominant gas constituent at helium-free average concentrations from about 50 (Gilsonite) to as much as 83 percent (petroleum coke). In all cases, the hydrogen concentration was lower than desired, ranging from about 20 percent (Gilsonite) down to as low as about 4 percent (petroleum coke). Greater hydrogen concentration is of interest because it directly contributes to reduced electrolysis power when linking CAVoR to a methanation/electrolysis system.

The relatively low hydrogen concentrations and the relatively high carbon dioxide concentrations (as well as high CO₂:CO ratio) were indicative of excess oxygen for the amount of available carbon, and also possibly indicated colder temperatures than required for optimum reforming kinetics. Operating experience showed that a portion of the carbonaceous matter was often evolved and recovered as an organic liquid that was collected in the condensate with water just before the injection of steam and oxygen were started. In many cases, this apparent pyrolysis during reactor pre-heating resulted in removal of roughly 20 percent of available carbon from the simulant, leading to an effectively higher oxygen:carbon ratio than was targeted for autothermal steam reforming. Therefore, higher temperature and reduced oxygen flow resulted in a substantial improvement in hydrogen production and overall steam reforming results. The continuous or long-operating-duration reactor described later ensures that any pyrolysis vapors formed as simulant is heated pass through the reforming zone, thereby maximizing recovery of hydrogen and minimizing water clean up requirements prior to electrolysis.

During each test, the organic matter within the reaction zone (upper section of the reactor) was virtually all reacted as indicated by the appearance of the residues and by the evolution of carbon-containing gases that were analyzed in the dry reformer exhaust streams.

It was observed during simulant preparation that only very small amounts of dark carbonaceous material would render the overall simulant mixture a dark color. Based on this observation and the measured carbon concentrations in the exhaust gas, the residue from the Gilsonite simulant contained virtually no carbonaceous matter. The oils sands residue was also virtually free of organic matter. The resulting residue consisted of montmorillonite clay mixed with oil-free sand. The petroleum coke residue appeared to contain a few residual particles of coarse organic matter from the feed as well as a small mass of low-density ash. The petroleum coke results shows that more-refractory carbonaceous feeds might require a longer reaction time than the 40 minutes used for the above experiments. The change in appearance of the oil-shale-containing asteroid simulant was not as distinct as the others except for color changes due to mineral transformations or oxidation of iron-containing compounds. All residues were easily removed from the reactor system using the pneumatic transfer system described above—no fusion or bridging was noted after any of the tests.

The results of the initial Phase I experiments using different organic constituents showed differences in both physical properties and reaction properties. For example, the Gilsonite feed was a fine powder that was readily mixed into the substrate to prepare simulant. Some care was required to prevent segregation of the fine powder from the coarser substrate when loading the reactor. The petroleum coke handled in a similar manner, but there were more coarse refractory particles in the petroleum coke feed. The oil sands were more difficult to blend with the montmorillonite clay substrate due to the sticky nature of the contained bitumen. However, a uniform blend was prepared and loaded into the reforming reactor. The oil shale was readily mixed with the clay substrate due to the similar particle size and angularity. The oil shale contained more fine material than the clay, so care was taken to avoid segregation during blending and loading into the reactor. Testing showed that the entire range of simulants could be successfully loaded, reacted, and removed from the Phase I apparatus. The anticipated characteristics of actual asteroid regolith (centimeter or smaller particles/weak aggregates, etc.) are consistent with the design of the Phase I batch reactor as well as a conceptual Phase II continuous flow reactor.

### Effect of Oxygen Ratio, Steam Ratio, and Temperature

Based on the results described in the previous section, reforming conditions were revised to simultaneously boost steam:carbon ratio (to help drive the reaction equilibrium), to increase temperature (to better favor steam reforming over oxidation), and to reduce the oxygen:carbon ratio (to maximize hydrogen formation while reducing consumption of oxygen to form carbon dioxide). Other individual Phase I experiments had been carried out to evaluate steam and temperature effects, but results from experiments carried out at a constant 0.5:1 O₂:C ratio were similar regardless of the range of steam ratios and temperatures used.

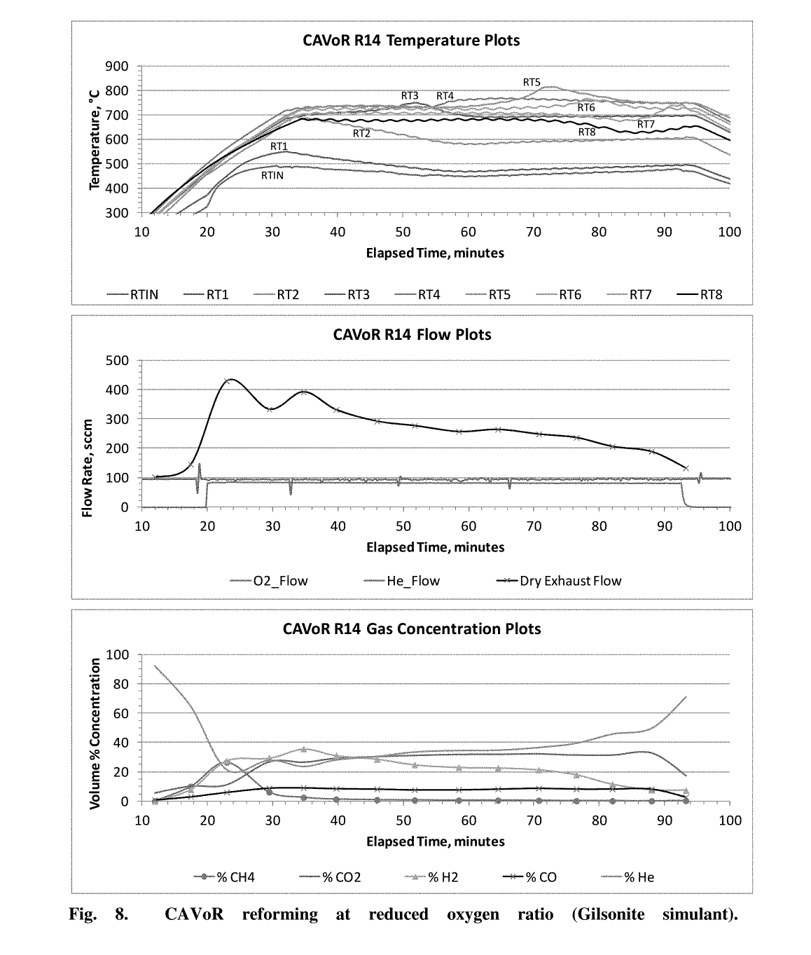

The results shown in FIG. 8 for experiment R14 (using a Gilsonite-containing simulant) clearly reflect a significant boost in hydrogen concentration as well as lower CO₂:CO ratio, resulting in a superior feed for a downstream methanation/electrolysis system. The results in FIG. 8 can be compared to those in FIG. 4 (R3; Gilsonite) to see the improvements brought about by operating with a reduced oxygen:carbon ratio and higher temperature. Typical simulant bed temperatures between about 700 and 800° C. were achieved during R14 versus 600 to 700° C. during R3. This, along with reduced oxygen flow, aided the thermochemical production of hydrogen.

During earlier experiments, the time during which steam and oxygen were injected was about 40 to 55 minutes for the nominal 40 minute operation. (Steam and oxygen were injected until the reaction exotherm passed thermocouple RT7, located just above the carbon pellet guard bed as described earlier). During experiment R14 at lower oxygen:carbon ratio, the run duration was 72 minutes. The time required to completely reform the simulant was greater than during other experiments carried out at higher oxygen:carbon ratios, likely as a result of the predominance of steam reforming over partial oxidation. The longer run time contributed to the addition of more total oxygen than desired, which reduced hydrogen production compared to the target conditions.

As during previous experiments, some pyrolysis occurred during heat up (note the rise in exhaust gas flow just before starting oxygen and steam). The spike in methane production (also via pyrolysis) corresponds to the release of heavier hydrocarbons collected as organic liquids. When discounting the small mass of unreacted simulant in the lowest section of the reactor, a reforming exhaust gas carbon yield of about 65 percent of that available in the feed simulant was accounted for by gas flow and analysis measurements (as carbon monoxide, carbon dioxide, and methane). An estimated additional 20 percent of the carbon contained in the simulant feed was accounted for in the organic matter in the condenser. It is likely that during pyrolysis, other non-condensable hydrocarbon gases such as ethane and higher alkanes were briefly evolved, which would account for remaining carbon released from the simulant.

This non-optimized experiment verified that on average about 18 percent of the total hydrogen required for methanation was produced by reforming of the simulant. The peak hydrogen concentration (about 47 percent by volume on a helium-free basis) generated during the earlier portion of the experiment would generate about 27 percent of the hydrogen needed for methanation. Earlier work by Pioneer on high-concentration organic feeds showed that about 40 percent of the required hydrogen can be produced by autothermal steam reforming (reducing electrolysis power requirements accordingly). Based on present modeling and past experience, the 40 percent value can be achieved for CAVoR upon optimization of operating conditions using the Phase II reactor system that minimizes loss of organic matter as higher hydrocarbons and condensable liquids.

### Effect of Reforming Rate

A limited number of experiments were run to determine the rate of oxygen and steam addition on reforming. Using a 1:1 steam:carbon ratio and a 0.5:1 oxygen:carbon ratio, the Gilsonite-containing simulant was successfully reformed using target reaction times of 20, 40, an 60 minutes (with actual times of 24, 50, and 69 minutes, respectively). In each case, a virtually carbon-free residue was obtained. Although there was a slight trend toward higher hydrogen production at the longer reaction times, results for each experiment were very similar. The results, along with others described above showed that the reformer is robust with respect to its performance over a wide range of conditions. The hardware is capable of producing high carbon yields even when operating outside optimum conditions.

### Sulfur Release and Capture During Reforming

The release of sulfur gases was monitored during the initial Phase I experimental program. A sulfur trap consisting of a bed of zinc oxide pellets (48 grams of Johnson Matthey HiFUEL 310) was installed prior to experiment R3. The sulfur trap was installed to both protect the exhaust gas oxygen sensor and to develop data related to removal of sulfur compounds under conditions of interest. Zinc oxide is effective at ambient temperature for high efficiency H₂S capture. However, its performance for trapping COS and organic sulfur compounds is not particularly good at ambient temperature. Zinc oxide is more effective for both H₂S and COS capture at elevated temperatures (above about 80° C. to 200° C. or greater).

Samples taken before and after the sulfur sorption column were analyzed during reforming experiments for each of the feed simulant types discussed above. In general, the results showed that H₂S capture was very high throughout the program with no change of the sorbent in the column. The results showed very little COS capture at ambient temperature. Results are shown in Table 13.

TABLE 12
CAVoR Sulfur Trap Performance.

| | | Pre Trap, | | Post Trap, | | | |
|---|---|---|---|---|---|---|---|
| Run | Simulant | Average ppm | | Average ppm | | % Capture | |
| # | Type | H₂S | COS | H₂S | COS | H₂S | COS |
| R4 | Gilsonite | 2415 | 12 | 4 | 11 | 99.8 | 11 |
| R6 | Oil Sands | 15,500 | 101 | 10 | 83 | 99.9 | 18 |
| R7 | Pet Coke | 2078 | 46 | 4 | 47 | 99.8 | 0 |
| R11 | Oil Shale | 2315 | 22 | 3 | 22 | 99.9 | — |
| R14 | Gilsonite | 1974 | 3 | 6 | 10 | 99.7 | — |
| | (low O2) | | | | | | |

The zinc oxide trap achieved H2S removal efficiencies of 99.7 to 99.9 percent. COS removal was negligible when considering variability in concentrations during testing and that the post-trap sample times did not exactly correspond with pre-trap sample times. Results for R14 (lower oxygen:carbon ratio and higher temperature) showed a substantial reduction in COS production compared to similar tests carried out at higher oxygen:carbon ratio and lower temperatures. The R14 sulfur results are more-representative of the distribution of sulfur in an optimized CAVoR system.

### Effect of Reforming on Magnetite Minerology

Carbonaceous chondrite asteroids may contain several percent of magnetite. As such, its interaction with reforming gases could impact the yields of hydrogen and oxygen. In addition, magnetite could represent an additional resource for oxygen recovery (a five percent magnetite concentration in asteroid regolith could yield about 1.4 percent oxygen via hydrogen reduction of magnetite to metallic iron with subsequent electrolysis of the product water). In advanced systems, the iron compounds could have value as an additional ISRU resource if recovered from reforming residues.

An experiment was carried out in which relatively high purity magnetite (F.J. Brodman & Co.; 60×100 mesh; 99.8% Fe₃O₄) was added to an oil-shale-containing simulant (experiment R12). The reforming experiment was conducted at a 2:1 steam:carbon ratio, 0.5:1 oxygen:carbon ratio for a nominal 40 minute reaction time at a target reactor shell temperature of 700° C. The hydrogen yield was somewhat greater than for another experiment with oil shale simulant (R11), but the temperature during R12 was higher.

Most of the magnetite residue from the reformer was separated from the clay substrate using a high-strength permanent magnet. The concentrated residue was then subjected to x-ray diffraction analysis. Results showed that the iron-bearing residue collected after reforming contained about 65 percent magnetite, 23 percent hematite, and 5 percent each of quartz and feldspar minerals (likely interlocked with a small portion of the clay residue). The x-ray diffraction results showed the conditions in the reformer were somewhat oxidizing overall. This is not surprising given that the initial Phase I batch reactor was configured such that the fixed location of inlet injection port above the asteroid simulant bed allow steam and oxygen to continue to pass over the spent residue above the downward-moving reaction zone. After removal of organic matter, the oxygenated steam passing through the residue above the reaction zone would result in oxidizing conditions.

In any case, the results show that magnetite, a potentially large constituent of carbonaceous chondrite asteroid regolith can be a reactive material that could tie up some oxygen if exposed to oxidizing conditions. The later continuous CAVoR auger reactor concept would more likely result in at least partial reduction of magnetite as an overall reducing condition would exist in the autothermal steam reforming zone. Asteroid regolith reforming residues that contain large amounts of reducible iron oxides could also be subjected to a separate hydrogen reduction step for recovery of an incremental additional amount of oxygen.

### Supplemental Oxygen Addition

A single experiment was conducted to determine whether supplemental oxygen injected in the lower portion of the reactor through a port located between thermocouples RT6 and RT7 could help to decompose organic matter produced by pyrolysis during pre-heating prior to injection of steam and oxygen. In most cases, steam and oxygen injection was started when thermocouple RT2 reached a temperature of 450° C. as the reactor and contents were heated. For this experiment, based on the appearance of pyrolysis liquids in previous condensate samples, supplemental oxygen at a flow rate of 40 sccm was initiated when thermocouple RT7 reached 400° C. (about 5 minutes before normal steam and oxygen injection). The supplemental oxygen flow was maintained for 12 minutes after starting the main steam and oxygen flows in an attempt to partially oxidize heavier organic compounds released as the lower portion of the simulant bed continued to heat.

The target oxygen flow was based on the typical amount of pyrolysis oil collected during previous experiments and its estimated contained carbon content. A conservative value of about one-half of the calculated oxygen requirement to convert carbon to carbon monoxide was used (40 sccm) in order to prevent possible reaction with the carbon pellet guard bed or possible channeling to the reactor exhaust and condenser system.

Results for the experiment showed no significant difference compared to experiment R12 with magnetite. No detectible exotherm was noted in the region of the supplemental oxygen injection port, and the overall gas production was similar to that produced in the absence of supplemental oxygen. The condenser samples appeared to be similar to those produced in the absence of supplemental oxygen.

The results showed that a higher flow of supplemental oxygen would be required to achieve partial oxidation of the small amount of pyrolysis liquids generated during pre-heating. However, the continuous reactor system developed during the course of the Phase I program would minimize production of pyrolysis liquids and would therefore eliminate the need for supplemental oxygen. As a result, no further testing was carried out on this concept.

### CAVoR Reforming-Methanation-Electrolysis Model

An Excel® computational model was developed to represent the CAVoR autothermal steam reforming system coupled to a downstream methanation-electrolysis system with net products including oxygen, methane, and water. Although other processes can be coupled to the reformer, the methanation-electrolysis system contains a number of technologies that have heritage in microgravity application on the ISS and can therefore reduce overall implementation risks.

The model was useful in establishing the expected stream compositions, material balances, heat balances, and electrolysis power inputs for a wide range of CAVoR organic compositions and overall feed rates. The model also helped to validate experimental results and to identify process improvement and optimization strategies during the Phase I program.

The model was adapted from earlier Pioneer work (Carrera et al., 2013) for a reforming system using a feed containing very little inorganic matter. The CAVoR model incorporated the material balance and heating requirements for the large majority of inorganic material that is fed with the relatively small amount of organic matter and water to the reforming system. The model was revised to accommodate carbonaceous asteroid regolith, which unlike typical high-organic feeds from space outpost wastes or similar feed, will produce excess water rather than require water to close the material balance.

The CAVoR model inputs include the desired process feed rate, asteroid regolith water content, organic matter content, and the C—H—O concentrations of the organic matter. The oxygen:carbon and steam:carbon molar ratios are specified along with the projected CO₂:CO molar ratio in the reformer gas (based on thermodynamic data or empirical data). With these inputs, the model calculates the reformer stream compositions as well as the stream compositions and material balances for integrated methanation-electrolysis systems. Inputs to the methanation module include the target Sabatier methanation reactor hydrogen excess above stoichiometric, the amount of dry Sabatier reactor methane recycled to the reactor along with unreacted hydrogen and carbon dioxide, and the concentration of hydrogen reporting to the final methane product (which is recovered and recycled during methane liquefaction). The methanation system inputs are derived from empirical data developed during earlier work at Pioneer (Carrera et al., 2013) in which a Sabatier system was successfully operated at a rate sufficient to process organic wastes for a lunar outpost crew of four to produce oxygen and methane. The model includes individual tabs for Reformer Balance, Sabatier Balance, Membrane Balance, Electrolyzer-Water Balance as well as tabs for thermal energy calculations and internal material balance checks.

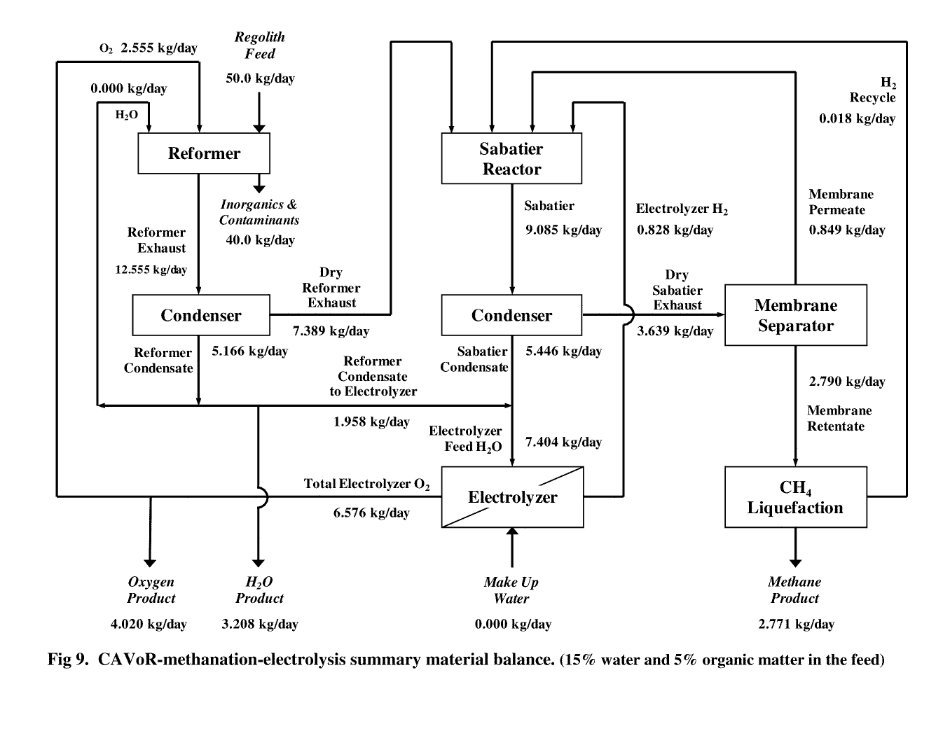

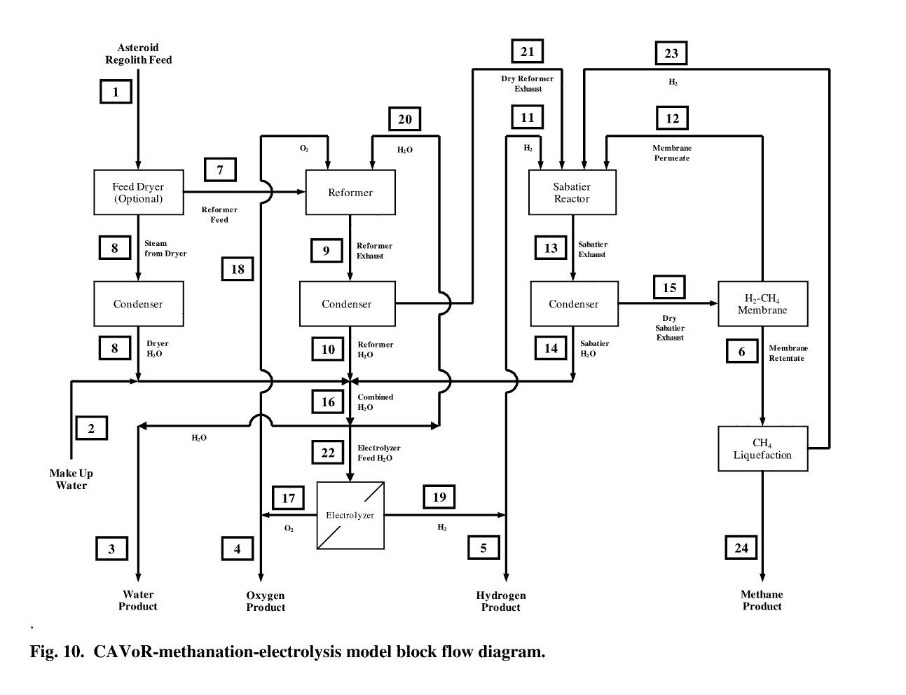

A baseline model representing a carbonaceous asteroid containing 15 weight percent water and 5 percent organic matter of a composition similar to kerogen was prepared using a regolith feed rate of 50 kilograms per day is summarized in FIG. 9. Table 14 shows the net inputs and outputs for this scenario including the primary internal process material mass flow rates. For the specified asteroid regolith composition, net products from 50 kg/day of regolith include 4.0 kg/day oxygen, 2.8 kg/day methane, and 3.2 kg/day water. FIG. 10 shows a detailed block flow diagram for the CAVoR reformer with downstream methanation and electrolysis units. The diagram identifies stream numbers that are listed in Table 14.

Note that the model includes provision for pre-drying feeds prior to reforming. This is only applicable to feeds that contain large amounts of moisture that can be released at relatively low temperatures without concurrent organic matter release. Pre-drying under those circumstances can reduce oxygen input that would otherwise be used to autothermally heat water contained in the feed stream when waste heat could be used instead.

The model results are based on calculation of carbon, hydrogen, and oxygen balances applied to the input conditions. For example, at the specified oxygen:carbon and steam:carbon ratios of 0.5:1 and 2:1, respectively, the dry reformer gas will contain about 58 percent H₂, 32 percent CO₂, and 11 percent CO (by volume). For this asteroid composition, the feed actually contains enough water for a 2.4:1 steam:carbon ratio. Therefore, no additional water is required. This gas composition contains about 37 percent of the hydrogen required for downstream methanation (resulting in a comparable electrolysis electrical energy savings compared to a combustion process in which all hydrogen for methanation would come from electrolysis).

Process sensitivity to the input parameters can be readily determined in support of system design and optimization. For example, a similar feed containing only carbon as the organic matter (versus the 8.7 and 8.4 weight percent hydrogen and oxygen for the above example) produces a reformer product gas containing about 43 percent hydrogen, 43 percent CO₂, and 14 percent CO. This product contains about 20 percent of the hydrogen required for methanation. The product suite shifts to 6.7 kg/day of oxygen, 3.3 kg/day of methane, and no water.

### Power and Mass Estimates

Rough estimates of CAVoR system power and mass requirements were made using the process model and preliminary evaluation of a continuous-flow reforming auger reactor with isolation valves and a large feed magazine and residue receiver. Although not intended to be comprehensive, this information helps to identify process operations for which a relatively high power or mass is projected. These operations were then addressed during further development. The rough estimates also help to establish the mass breakeven time for the delivered hardware against the mass of products generated over time.

As a starting basis, the 50 kg/day process unit described above was used for initial evaluation. Some savings at larger scale would be realized, but this rate represents a conservative starting point for continued development. The reformer section requires thermal power input as detailed in the Table 15. Much of this required heat input can be obtained through recovery of heat from the hot reformer gases (about 380 watts thermal) and the solid residue (about 350 watts thermal). A band heater around the reaction zone provides any necessary make up heat to sustain the reaction at target temperatures.

TABLE 14
Thermal Power Input Summary for 50 kg/day CAVoR Reformer.

| Regolith, | 1) | Heat Ice in Regolith from −73 to 0° C. | 13.4 | |
|---|---|---|---|---|
| Organics, | 2) | Melt Contained Ice at 0° C. | 29 | W |
| H₂O, and O₂ | 3) | Heat Contained Water from 0 to 100° C. | 36 | W |
| Feed Heating | 4) | Boil Contained Water at 100° C. | 197 | W |
| | 5) | Heat Contained Steam from 100 to 700° C. | 112 | W |
| | 6) | Heat Solid Organic Matter in Regolith from −73 to 78° C. | 6 | W |
| | 7) | Melt Contained Organics at 78° C. | 4 | W |
| | 8) | Heat Contained Liquid Organic Matter from 78 to 218° C. | 6 | W |
| | 9) | Vaporize Contained Organic Matter at 218° C. | 16 | W |
| | 10) | Heat Vaporized Organic Matter from 218 to 700° C. | 34 | W |
| | 11) | Heat Inorganic Matter from −73 to 700° C. | 494 | W |
| | 12) | Heat Oxygen from 20 to 700° C. | 21 | W |
| | | Total Reformer Regolith & O₂ Heating Power | 969 | W |

Note:
values are based on 15% water, 5% organic matter at about 83% C, 8.7% H, and 8.3% O.
The corresponding methanation system (operating as described in the model above) produces about 330 watts of thermal power via the hot reactor exhaust gas. Table 16 summarizes heat generation for the 50 kg/day CAVoR system (10 kg/day water plus organic matter).

TABLE 15
Thermal Power Input Summary for
50 kg/day CAVoR Methanation System.

| | | | Thermal |
|---|---|---|---|
| | | | Power, |
| Operation | Description | | Watts |
| Sabatier | 1) | Heat Sabatier Feed Gas from | 137 |
| Reaction | | 20 to 450° C. | |
| | 2) | Sabatier Reaction (CO + | −384 |
| | | CO2 + H2 = CH4 + H2O) | |
| | | Total Sabatier Reaction | −246 |
| | | Thermal Power | |
| Sabatier | 1) | Indirect Heat Exchange by | −99 |
| Reactor | | Reactor Feed Gas | |
| Cooling | | | |
| Sabatier | 1) | Cool Exhaust Gas from | −66 |
| Exhaust | | 450 to 100° C. | |
| Gas | 2) | Condense Sabatier Exhaust | −143 |
| Cooling | | Water at 100° C. | |
| | 3) | Cool Dry Sabatier Exhaust | −5 |
| | | Gas from 100 to 20° C. | |
| | 4) | Cool Sabatier Condensate | −21 |
| | | from 100 to 20° C. | |
| | | Total Sabatier Exhaust | −235 |
| | | Cooling Power | |
| | | Total Operating Power | −334 |

Note:
(+) = heat input required;
(−) = waste heat available

Some additional thermal power inputs (supplied by waste heat or by electrical power) are required for maintaining temperatures for instrumentation, valves, and other key process support units operating in a cold environment. These are expected to be minor compared to the process thermal needs detailed above.

The primary electrical power requirement to support the CAVoR reforming-methanation- electrolysis system is electrolysis power, which for the 50 kg/day asteroid regolith feed rate and kerogen composition is about 1.4 kW (at 100 percent efficiency; about 1.75 kW at 80 percent efficiency). Note that the alternate case discussed in the previous section that uses pure carbon as organic matter contained in asteroid regolith (i.e. hydrogen-free feed), the electrolysis power rises to about 2.1 kW at 100 percent efficiency. Additional electrical power inputs will be required for instrumentation, pumps, actuators, and other ancillary operating hardware. These are expected to be minor relative to electrolysis needs,.

A rough estimate of mass using the continuous flow auger reactor system described below is approximately70 kilograms. By comparison, the estimated flight-ready reformer mass for Pioneer's Lunar Organic Waste Reformer (LOWR) was 28 kg for a high-moisture, organic matter feed rate of about 6 kg/day. The LOWR reformer was substantially different than the one described here for asteroid regolith processing due to the very small amount of inorganic matter in the LOWR feed and its high-oxygen organic matter feed content. As a result, the organic matter for LOWR was reacted in a fixed location with feed readily transported with only minor compression force into the reaction zone as organic matter was consumed (unlike the continuous CAVoR system which requires that the regolith containing minority organic matter be actively transported through the reaction zone).

The mass for the methanation-electrolysis unit is estimated at about 85 kg based on earlier Pioneer work for a similar-scale unit as part of the Lunar Organic Waste Reformer (Carrera et al., 2013). Although the reformer for that application was significantly different than that required for asteroid regolith processing, the downstream methanation-electrolysis size and configuration is similar. Most of the mass is associated with electrolysis, oxygen liquefaction, and methane liquefaction.

For a preliminary, rough total system mass estimate of 200 kg (with allowance for ancillary hardware including power supplies, framework, avionics, etc,) and the projected yield of 10 kg/day of combined water, methane, and oxygen product, a breakeven time of only 20 days is projected. Breakeven time is defined as the time at which the accumulated mass of product equals that of the processing hardware. This preliminary evaluation shows the tremendous potential for launch cost savings and in-space propellant production in support of advanced spaceflight.

Given the relatively low duty on sealing valves and limited number of rotating seals, the consumables requirements for a complete CAVoR system are expected to be low, resulting in a very high process leverage against the mass of products generated. Leverage is defined as the ratio of product mass to consumables mass.

### Continuous Reforming Auger Reactor Design

From the results of initial Phase I testing, modeling, and process design, a conceptual continuous flow auger reactor was identified as an ideal candidate to support the further development, scale-up, and implementation of the CAVoR reformer and downstream process units. The conceptual auger reactor incorporates the following features.
accommodates continuous or long-duration operation to minimize start up transients via the use of large feed magazines and residue receivers to minimize the duty on isolation valves,
enables efficient indirect heat exchange from the hot reformer exhaust gases and regolith residue to preheat fresh asteroid regolith, which is aided by the mixing of solids by the auger,
allows independent adjustment of auger speed and oxygen injection rates to maximize utilization and performance over a wide range of feed characteristics,
incorporates a band heater to concentrate any required make up heat in the reaction zone, thereby reducing heat losses,
requires low torque to move granular asteroid regolith through the preheating, reaction, and cooling zones—torque monitoring and control can be used to ensure proper particle packing in the microgravity environment, and a simple tensioned plunger can be used to provide the required force to convey feed from the magazine into the auger and to provide flow resistance into the residue receiver,
all moisture and organic matter that is vaporized during preheating passes through the fixed reaction zone, ensuring maximum conversion and recovery of organic matter as the desired H₂, CO, CO₂, and H₂O products,
the primary feed magazine and residue receiver seals consist of non-rotating, flange or clamp-type seals amenable to straightforward motion controls,
only the auger shaft requires a rotating seal, which can be integrated with a small sweep gas flow and pressure equilibration system to minimize losses, and
the areas around the two isolation valves that are required to be closed during resupply of the feed magazine and residue receiver can be cleared of particulates prior to operation using integrated internal flow control “flappers” in combination with small pneumatic pulses or induction of small ballistic trajectories.

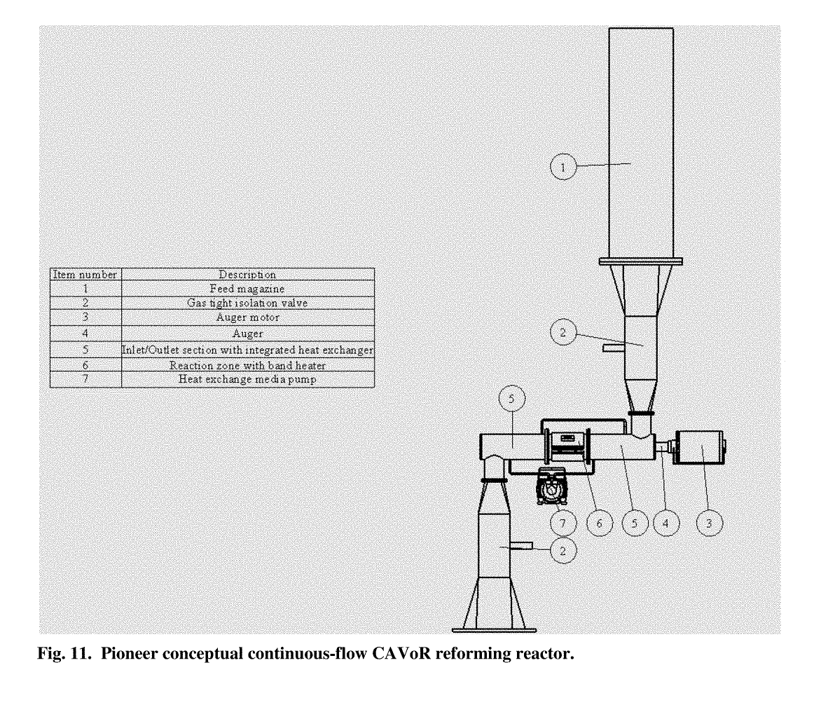

The conceptual continuous flow CAVoR reactor is depicted in the FIG. 11.

The continuous auger reactor shown in FIG. 11 was drawn in SolidWorks® using available industrial components that are not entirely representative of a flight-like unit. However, many of the main features highlighted in the bullets above are shown in the drawing. A large feed magazine (and a comparable residue discharge receiver—not shown) allow for long periods of operation between depressurization and refill procedures, thereby reducing the duty on the gastight valves and flanges that connect the magazine and receiver to the reactor body. Internal compression devices (not shown) are used to provide sufficient force to place fresh regolith into the auger feed zone and to supply sufficient back pressure on the auger discharge to prevent regolith from freely passing through the auger in microgravity conditions.

Depending on the magazine and receiver volumes, the reactor could be paused during feed/discharge vessel swaps or the entire system could be cleared and restarted. The feed magazine and receiver have simple wide throats to facilitate material transfers.

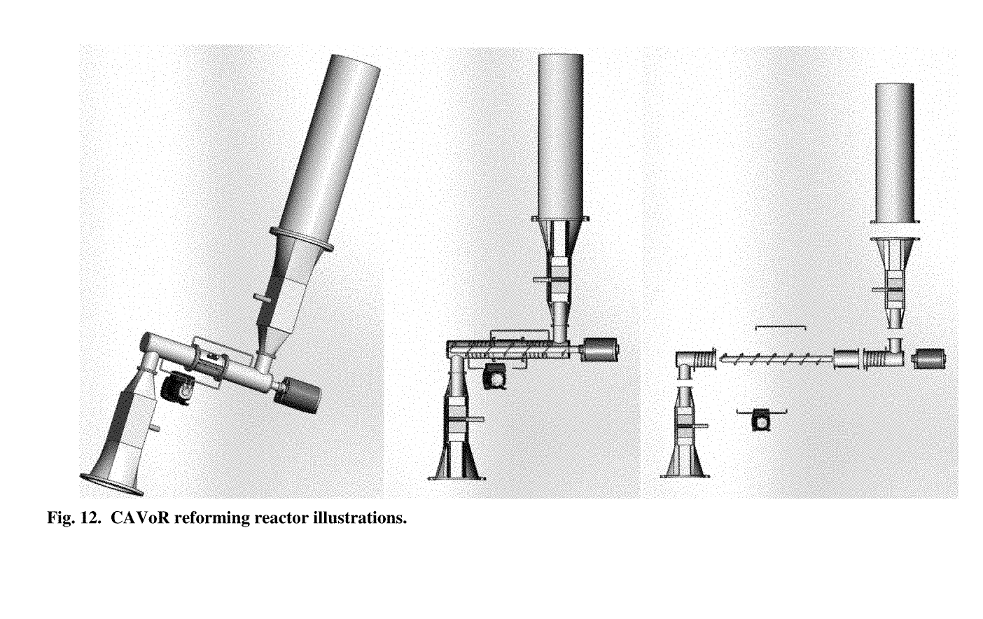

A heat recovery and return system is depicted schematically around the residue discharge zone and feed regolith zones downstream and upstream of the center reaction zone. Several options are available for the transfer of thermal power from regolith residue and process gases to minimize the net power input to the CAVoR reformer. For example, indirect heat exchange to transfer thermal energy from the CAVoR reformer exhaust gases and methanation exhaust gases to regolith feed can be accomplished using the process fluids as proceed to their respective condenser system. Additional indirect heat exchange from the spent regolith to the feed can be accomplished recirculating CAVoR-derived heat transfer media such as carbon dioxide or nitrogen, which are in large part compatible with the range of temperatures expected in the reforming process. Alternatively, optimized heat transfer media can be used in a closed-loop system for heat transfer within specified temperature ranges. FIG. 12 shows additional views of the conceptual CAVoR continuous auger reactor system.

Small amounts of non-reactive gases can be used for pressure equalization during feed magazine and receiver exchanges. Gases such as CO₂ or N₂ can be derived from the CAVoR processing to minimize Earth supplied consumables. The majority of pressure equalization gases can be recovered and stored prior to each gas-lock operation, but some gas will be lost in a manner similar to operation of air locks on ISS.

### Terrestrial Oil Shale Processing

Methods such as those described above are applied to recovery of volatile and carbonaceous matter from terrestrial oil shale deposits. In terrestrial applications, the autothermal steam reforming conditions are adjusted to provide nearly complete removal and recovery of carbonaceous matter with simultaneous release of hydrogen, carbon monoxide, carbon dioxide, and water. The oxygen:carbon ratio and steam:carbon ratio are adjusted based on any specific oil shale composition to provide optimum temperatures and gas compositions to maximize release of valuable condensable organic matter. In this application, oxygen is supplied in the form of pure oxygen, oxygen-enriched air, and air. After recovering condensable organic matter for use as fuels or chemicals, non-condensable gases including hydrogen, carbon dioxide, and carbon monoxide are used directly as fuel or are fed to additional processing to produce methane, methanol, and other synthesis products.

### Terrestrial Coal Waste, Municipal Waste, and Contaminated Soil Processing

Methods such as those described above are applied to recovery of volatile and carbonaceous matter from coal wastes recovered from disposal sites. The autothermal steam reforming conditions are adjusted to provide nearly complete removal and recovery of carbonaceous matter with simultaneous release of hydrogen, carbon monoxide, carbon dioxide, and water. The oxygen:carbon ratio and steam:carbon ratio are adjusted based on any specific waste composition to provide optimum temperatures and gas compositions to maximize release of carbonaceous matter. The autothermal steam reforming gas composition is optimized to minimize or maximize production of condensable organic compounds based on market conditions. In this application of the device, oxygen is supplied in the form of pure oxygen, oxygen-enriched air, and air. After recovering condensable organic matter for use as fuels or chemicals, non-condensable gases including hydrogen, carbon dioxide, and carbon monoxide are used directly as fuel or are fed to additional processing to produce methane, methanol, and other synthesis products.

### REFERENCES

The following citations are incorporated by reference.
American Petroleum Institute Petroleum HPV Testing Group, “Crude Oil Category—Category Assessment Document”, High Production Volume (HPV) Chemical Challenge Program, Submitted to the U.S. EPA, Jan. 14, 2011.
Berggren, Mark, Robert Zubrin, James Kilgore, Cherie Wilson, Peter Jonscher, Sam Michael, Heather Rose, and Nick Jameson, “Lunar Materials Handling System”, NASA JSC SBIR Phase II Contract NNJ07JA22C, Final Report, Pioneer Astronautics, Apr. 26, 2010.
Binzel, Richard P., Schelte J. Bus, Thomas H. Burbine, and Jessica M. Sunshine, “Spectral Properties of Near-Earth Asteroids: Evidence for Sources of Ordinary Chondrite Meteorites”, Science, vol. 273, pp. 946-948, Aug. 16, 1996.
Bose, Kunal and Jibamitra Ganguly, “Thermogravimetric study of the dehydration kinetics of talc”, American Minerologist, Vol. 79, pp. 692-699, 1994.
Burman, G. R. and E. E. Romagosa, “Gilsonite Resin, Its Production and Utilization”, Preprint No. 90-29, SME Annual Meeting, Salt Lake City, Utah, Feb. 26-Mar. 1, 1990.
Carrera, Stacy, Robert Zubrin, Mark Berggren, Aaron Hale, James Siebarth, and James Kilgore, “Multi Cell Thermal Battery”, NASA JSC Phase II Contract NNX10RA18C, Final Report, Pioneer Astronautics, Apr. 22, 2011.
Carrera, Stacy, Robert Zubrin, Peter Jonscher, Chris Hinkley, and Mark Berggren, “Lunar Organic Waste Reformer”, NASA Glenn Research Center SBIR Phase II Contract No. NNX11CA76C, Final Report, Pioneer Astronautics, Jun. 13, 2013.
Brown, G. H. and E. T. Montgomery, “Dehydration of Clays”, Technologic Papers of the Bureau of Standards, No. 21, U.S. Government Printing Office, Apr. 25, 1913.
Davis, Donald R., Alan L. Friedlander, and Thomas D. Jones, “Role of Near-Earth Asteroids in the Space Exploration Initiative”, Chapter in Resources of Near-Earth Space, Lewis, Matthews, and Guerrieri, Editors, University of Arizona Press, Tucson and London, 1993.
Ehrenfreund, Pascale, Daniel P. Glavin, Oliver Botta, George Cooper, and Jeffery L. Bada, “Extraterrestrial amino acids in Orgueil and Ivuna: Tracing the parent body of CI type carbonaceous chondrites”, Proceedings of the National Academy of Sciences of the United States of America, vol. 98, no. 5, pp 2198-2141, Feb. 27, 2001.
Kerridge, John F., “Origins of Organic Matter in Meteorites”, Proceedings of the National Institute of Polar Research, vol. 6, pp. 293-303, 1993.
Lewis, John S. and Melinda L. Hutson, “Asteroidal Resource Opportunities Suggested by Meteorite Data”, Chapter in Resources of Near-Earth Space, Lewis, Matthews, and Guerrieri, Editors, University of Arizona Press, Tucson and London, 1993.
Nichols, Charles R., “Volatile Products from Carbonaceous Asteroids”, Chapter in Resources of Near-Earth Space, Lewis, Matthews, and Guerrieri, Editors, University of Arizona Press, Tucson and London, 1993.
Oil Sands Discovery Centre, “Facts about Alberta's oil sands and its industry”, Oil Sands Discovery Center, www.oilsandsdiscovery.com, 2015.
O'Neill, Gerard K., “Space Resources and Space Settlements”, Billingham, Gilbreath, and O'Leary, Editors, NASA SP-428, 1979.
Pasek, M. A., V. D. Smith, and D. S. Luaretta, “A Quantitative NRM Analysis of Phosphorus in Carbonaceous and Ordinary Chondrites”, Lunar and Planetary Science XXXV Conference, 2004.
Plows, Fiona L., Jamie E. Elsila, Richard N. Zare, and Peter R. Buseck, “Evidence that polycyclic aromatic hydrocarbons in two carbonaceous chondrites predate parent-body formation”, Geochimica et Cosmochimica Acta, Vol. 67, No. 7, pp. 1429-1436, 2003.
Septhon, Mark A., “Organic compounds in carbonaceous meteorites”, Nat. Prod. Rep., vol. 19, Royal Society of Chemistry, pp. 292-311, 2002.
Stanfield, K. E., I. C. Frost, W. S. McAuley, and H. N. Smith, “Properties of Colorado Oil Shale”, Report of Investigations 4825, U.S. Department of the Interior, Bureau of Mines, 1951.
Tiwari, Pankaj, Kyeongseok Oh, and Milind Deo, “Oil Shale Pyrolysis: Characterization and Compositional Analysis”, 29^{th} Oil Shale Symposium, Colorado School of Mines, 2009.
Zubrin, Robert, Peter Jonscher, and Sam Michael, “Counterflow Regolith Heat Exhanger”, NASA KSC SBIR Phase I Contract NNX09CE75P, Final Report, Pioneer Astronautics, Jul. 22, 2009.

What is claimed is:

1. A device to extract and recover water and organic matter to support propellant production, breathing gas, and life support comprising:
a) Recovering water from asteroid regolith,
b) Recovering carbonaceous material from asteroid regolith,
c) Electrolyzing recovered water to form hydrogen and oxygen, and;
d) Converting the carbonaceous material in an autothermal reaction with steam and oxygen-containing gas to produce carbon monoxide, hydrogen, carbon dioxide and water.

2. The device of claim 1 where an auger reactor is used, allowing for nearly continuous operation and allowing for residual heat from the reactor discharge to be transferred to the gas feeds and fresh solid feed

3. The device of claim 1 where two batch reactors are used, allowing residual heat from one autothermal reactor to be indirectly exchanged to pre-heat steam, oxygen-containing gas, and regolith fed to the second reactor.

4. The device of claim 1 where further processing would be carried out to manufacture organic compounds and oxygen from the reaction products.

5. The device of claim 1 where production of clean syngas suitable as feed for downstream processes that are integrated to produce fuels, plastics, and other organic compounds along with oxygen and water.

6. The device of claim 1 where further processing is selected from the steps of reverse water gas shift (RWGS) to convert CO₂ to CO and to adjust the H₂:CO ratio, Sabatier/electrolysis to produce methane and oxygen, methanol synthesis (for fuel or subsequent olefins, plastics, and fuels synthesis, Fischer-Tropsch reactions to produce hydrocarbon fuels, or direct olefins synthesis from syngas).

7. The device of claim 2 where additional useful heat recovery can be obtained by indirect exchange of the hot exhaust gases to the asteroid feed material or to other feeds such as oxygen and water.

8. The device of claim 1 comprising a continuous flow auger reactor,

9. The device of claim 1 refining the mass, volume, and power requirements for a flight-like system including methanation-electrolysis at processing scales of interest for in-space resource utilization,

10. The device of claim 1 comprising integration of regolith excavation, handling, feeding, discharging, and collection/transport of products.

11. The device of claim 1 where gaseous contaminants are removed and potentially valuable condensed and residual solid byproducts are recovered, including the processed regolith that can be used as a material for fabrication of structures and mechanical components via additive manufacturing and advanced formation technologies.

12. The device of claim 1 where additional oxygen yield is obtained by reduction of iron oxide compounds contained in asteroid regolith by hydrogen or carbon monoxide to lower iron oxides or iron metal with concurrent production of water or carbon dioxide, which are processed to recover their contained oxygen content.

13. A device to extract and recover water and organic matter to support water production, fuels production, and chemicals production from high-ash coal, coal processing wastes, oil shale, municipal wastes, industrial wastes, and renewable resources and their byproducts comprising:
a) Thermally recovering water from low-grade, unconventional terrestrial resources and waste materials containing high concentrations of inorganic matter,
b) Recovering carbonaceous material from the above-stated resources and waste materials, and;
c) Converting the carbonaceous material in an autothermal reaction with oxygen-containing gas and steam to produce carbon monoxide, hydrogen, carbon dioxide and water.

14. The device of claim 13 where an auger reactor is used, allowing for nearly continuous operation and allowing for residual heat from the reactor discharge to be transferred to the gas feeds and fresh solid feed.

15. The device of claim 13 where two batch reactors are used, allowing residual heat from one autothermal reactor to be indirectly exchanged to pre-heat steam, oxygen-containing gas, and solid-phase resource fed to the second reactor.

16. The device of claim 13 where further processing would be carried out to manufacture organic compounds from the reaction products.

17. The device of claim 13 where production of clean syngas suitable as feed for downstream processes that can be integrated with the system to produce fuels, plastics, and other organic compounds along with water.

18. The device of claim 13 where further processing is selected from the steps of reverse water gas shift (RWGS) to convert CO₂ to CO and to adjust the H₂:CO ratio, Sabatier reaction to produce methane, methanol synthesis (for fuel or subsequent olefins, plastics, and fuels synthesis, Fischer-Tropsch reactions to produce hydrocarbon fuels, or direct olefins synthesis from syngas).

19. The device of claim 13 where additional useful heat recovery can be obtained by indirect exchange of the hot exhaust gases to fresh feed material or to other process feeds such as oxygen, air, and water.

20. The device of claim 13 comprising a continuous flow auger reactor,

21. The device of claim 13 including methanation and fuels synthesis at processing scales of interest for terrestrial resource and waste utilization,

22. The device of claim 13 comprising integration of resource excavation, handling, feeding, discharging, and collection/transport of products.

23. The device of claim 13 where oil shale is processed in a manner that releases condensable volatile organic matter concurrent with production of hydrogen, carbon monoxide, carbon dioxide, and water and where the condensable organic matter is collected as a fuel and chemical product.

24. The device of claim 13 where gaseous contaminants are removed and potentially valuable byproducts are recovered.

25. The device of claim 13 where residues from terrestrial processing are rendered sterile and made suitable for further separations, byproduct recovery, and disposal.
